(19) United States
(12) Patent Application Publication
(10) Pub. No.: US 2020/0147038 A1
(43) Pub. Date: May 14, 2020

(54) ASSESSING AND TREATING PSYCHEDELIC-RESPONSIVE SUBJECTS

(71) Applicant: Eleusis Benefit Corporation, PBC, New York, NY (US)

(72) Inventors: Suzanne RUSS, Dickinson, ND (US); Shlomi RAZ, Brooklyn, NY (US); Neiloufar FAMILY, Brooklyn, NY (US)

(21) Appl. No.: 16/606,559

(22) PCT Filed: Apr. 20, 2018
(86) PCT No.: PCT/US2018/028614

§ 371 Date: Oct. 18, 2019

Related U.S. Application Data
(60) Provisional application No. 62/487,718, filed on Apr. 20, 2017.

(51) Int. Cl. A61K 31/36; A61K 45/06; A61K 31/135

(52) U.S. Cl. CPC A61K 31/36; A61K 31/135; A61K 45/06

(57) ABSTRACT

The invention features methods of identifying a subject as being likely to have a positive therapeutic response to a psychedelic agent. Methods of the invention also include administering a psychedelic agent to a subject (e.g., a subject that has been identified as likely to respond positively thereto) to improve mental or physical well-being in the subject (e.g., by treating stress, anxiety, addiction, depression, compulsive behavior, by promoting weight loss, by improving mood, by treating or preventing a condition (e.g., psychological disorder), or by enhancing performance).

### BACKGROUND OF THE INVENTION

Therapeutic benefits of psychedelic drugs, such as lysergic acid diethylamide and psilocybin, are being actively researched in humans. Studies in healthy volunteers have shown long-term increases in trait optimism (Carhart-Harris et al., Psychological Medicine 2016, 46:1379-1390), well-being (Id. and Griffiths et al., Psychopharmacology 2011, 218:649-665), and openness (Carhart-Harris et al., Psychological Medicine 2016, 46:1379-1390; MacLean et al., Journal of Psychopharmacology 2011, 25:1453-1461), and studies in patients have found long-term improvements in obsessive compulsive disorder (Moreno et al., Journal of Clinical Psychiatry 2006, 67:1735-1740), tobacco addiction (Garcia-Romeu et al., Current Drug Abuse Reviews 2014, 7:157-164), alcoholism (Krebs and Johansen, Psychopharmacology 2012, 26.7:994-1002; Bogenschutz et al., Journal of Psychopharmacology 2015, 29.3:289-299), narcotic addiction (Savage and McCabe, Psychiatry 1973, 28.6:808-814), depression and anxiety related to diagnosis of a life-threatening or terminal illness (Grob et al., Archives of General Psychiatry 2011, 68:71-78; Griffiths et al., Journal of Psychopharmacology 2016, 30(12):1181-1197; Ross et al., Journal of Psychopharmacology 2016, 30(12):1165-1180), and depression (Carhart-Harris et al., The Lancet Psychiatry 2016; Sanches et al., Journal of Clinical Psychopharmacology 2016, 36:77-81) after treatment with psychedelics. These long-term effects can endure for several months, if not years, after the compound has been metabolized and excreted from the body.

Such outcomes challenge conventional models on pharmacological interventions for mental illness, as well as models on the nature of psychological traits such as personality, which should be relatively insensitive to change by adulthood. Despite these impressive outcomes, no systematic approach exists for identifying individuals for whom psychedelic treatment is likely to produce an optimal result. These findings demand further research and raise important questions about mechanisms underlying positive response to psychedelic treatments and how they can be predicted a priori. In line with this incentive, the National Institute of Mental Health has set forth Research Priority IIB, which seeks to “develop biomarkers and assessment tools to predict . . . intervention response across diverse populations.” Thus, there is a clear need in the field for methods to predict a subject's response to psychedelic therapy (e.g., lysergic acid diethylamide or psilocybin therapy).

### SUMMARY OF THE INVENTION

The present invention provides methods of improving mental or physical well-being (e.g., by treatment of stress, anxiety, addiction, depression, psychological disorders, or behavioral disorders) by identifying a course of therapy for a subject, e.g., based on personality state or trait predictors. Therapies described herein include pharmacological therapies (e.g., psychedelic agents, e.g., 5-HT_{2A} agonists, dissociative agents, or empathogenic agents), psychotherapies (e.g., behavioral therapies), and combinations thereof (e.g., complex drug and non-drug combination treatment regimens). The invention includes methods of administering a psychedelic agent to a subject that has been identified as likely to have a positive therapeutic response to the psychedelic agent. Alternatively, the invention provides methods for reducing exposure to a psychedelic agent for therapeutic benefit in subjects that are not likely to benefit from the psychedelic agent.

In one aspect, the invention features a method of improving mental or physical well-being of a subject, the method including: (i) providing a subject, wherein based on a score of one or more predictors in the subject, the subject has been identified as likely to have a positive therapeutic response to a psychedelic agent (e.g., a 5-HT_{2A} agonist (e.g., lysergic acid diethylamide or psilocybin), a dissociative agent (e.g., ketamine), or an empathogenic agent (e.g., 3,4-Methylenedioxymethamphetamine (MDMA)); and (ii) following step (i), administering to the subject the psychedelic agent. In some embodiments, the method is for treating stress in the subject, treating anxiety in the subject, treating addiction in the subject, treating depression in the subject, or treating a compulsive behavior in the subject. In some embodiments, the subject is obese, and the method comprises promoting weight loss in the subject. In some embodiments, the method is for treating a condition in a subject, improving the mood of a subject, or enhancing the performance of a subject. In some instances, the condition is a psychological disorder.

In some embodiments of the invention, the one or more predictors include one or more trait predictors, such as a measure of absorption and/or a measure of mental barriers. In some instances, a high measure of absorption is positively correlated with a positive therapeutic response. For example, a positive therapeutic response may be predicted if a subject has a high measure of absorption and a high measure of surrender; a high measure of absorption and a low measure of mental barriers; or a high measure of absorption, a high measure of surrender, and a low measure of mental barriers. Additionally or alternatively, a high measure of mental barriers is inversely correlated with a positive therapeutic response.

In some embodiments, the one or more predictors include one or more state predictors. In some instances, the one or more state predictors are selected from the group consisting of a measure of distress, a measure of preoccupation, and a measure of surrender. In some embodiments, a high measure of distress is positively correlated with a positive therapeutic response. For example, a positive therapeutic response may be predicted if a subject has a high measure of distress and a high measure of surrender. Additionally or alternatively, a high measure of preoccupation may be inversely correlated with a positive therapeutic response. In some embodiments, a high measure of surrender is positively correlated with a positive therapeutic response.

In some embodiments, the one or more predictors include one or more habits (e.g., frequency of meditation and/or frequency of cannabis use). In some embodiments, frequency of meditation and/or frequency of cannabis use are positively correlated with a positive therapeutic response.

In some embodiments, the one or more predictors are selected from the group consisting of a measure of absorption, a measure of mental barriers, a measure of meditation frequency, a measure of distress, a measure of preoccupation, a measure of surrender, and a measure of dread.

In some embodiments of any of the preceding methods, the score can be a composite score of at least two, at least three, at least four, at least five, at least six, at least seven of the predictors. The composite score can be a weighted composite score. For example, each predictor can be weighted based on its explanatory power on positive therapeutic response to the psychedelic agent. Explanatory power can be determined, e.g., by a regression model, such as a hierarchical linear multiple regression.

In another aspect, the invention features a method of improving the mental well-being of a subject, the method comprising: (i) providing a subject, wherein the subject has been identified as likely to have a positive therapeutic response to a psychedelic agent based on a high measure of: absorption; identity distress; or surrender; and (ii) following step (i), administering to the subject the psychedelic agent.

In another aspect, the invention features a method of reducing the risk of developing a psychological disorder in a subject in need thereof, the method comprising: (i) providing a subject, wherein the subject has been identified as likely to have a positive therapeutic response to a psychedelic agent based on a high measure of: absorption; identity distress; or surrender; and (ii) following step (i), administering to the subject the psychedelic agent.

In some embodiments of any of the preceding aspects, the subject has been identified as likely to have a positive response to a psychedelic agent based on a low measure of: mental barriers; and/or preoccupation.

In some embodiments of any of the preceding methods, the psychedelic agent is administered as an adjunctive therapy, wherein the subject is being treated with, has been treated with, or is going to be treated with a psychotherapy. In some embodiments, step (ii) further comprises treating the subject with a psychotherapy.

The psychotherapy may be a behavioral psychotherapy (e.g., talk therapy). In some instances, the psychotherapy includes existential or humanistic therapy. In some instances, the psychotherapy includes self-actualization therapy. In some embodiments, the subject is instructed to meditate (e.g., during treatment with a psychedelic therapy).

In some embodiments of any of the preceding aspects, step (ii) occurs within a psychotherapeutic setting, such as a specialized treatment facility.

In some embodiments of any of the preceding aspects, the psychological disorder is selected from the group consisting of a depressive disorder, an anxiety disorder, an addiction, or a compulsive behavior disorder. In some instances, the psychological disorder is a depressive disorder (e.g., major depression, melancholic depression, atypical depression, or dysthymia). The depressive disorder may be associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses, any of which may be treated using any of the preceding methods described herein. The psychological disorder may be an anxiety disorder (e.g., end of life anxiety, generalized anxiety disorder, panic disorder, social anxiety, post-traumatic stress disorder, acute stress disorder, obsessive compulsive disorder, or a social phobia). The psychological disorder may be an addiction (substance abuse or an eating disorder). The psychological disorder may be a compulsive disorder (e.g., a primary impulse-control disorder or an obsessive-compulsive disorder). In some embodiments, the methods of the invention can be used to treat a symptom of the psychological disorder (e.g., a psychosomatic symptom or a somatic symptom (e.g., chronic pain, anxiety disproportionate to severity of physical complaints, pain disorder, body dysmorphia, conversion, hysteria, neurological conditions without identifiable cause, or psychosomatic illness)). In some instances, the psychological disorder is a repetitive body-focused behavior (e.g., a tic disorder, e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

In some embodiments, the psychedelic agent is selected from lysergic acid diethylamide, psilocybin, and pharmaceutically acceptable salts thereof. In some embodiments, the psychedelic agent is a 5-HT_{2A} agonist (e.g., LSD, psilocybin, DOI (±)-1-(2,5-dimethoxyphenyl)-2-aminopropane hydrochloride; (R)-DOI ((R)-1-(2,5-dimethoxy-4-iodophenyI)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine); ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III), or a pharmaceutical acceptable salt thereof).

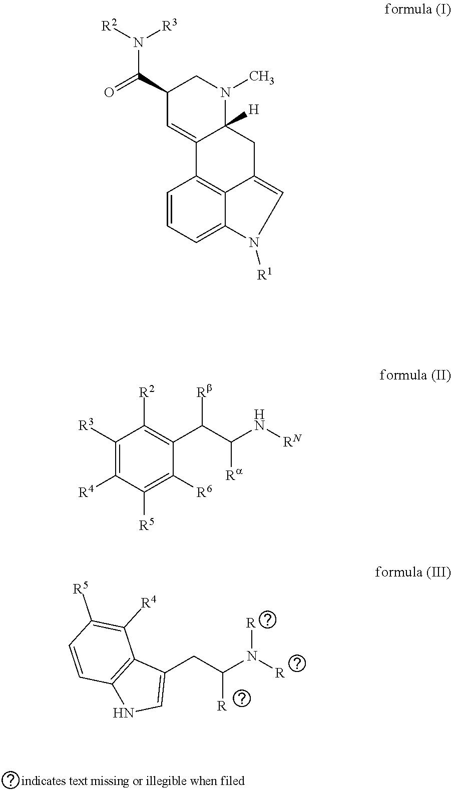

In other embodiments, the psychedelic agent is a dissociative agent (e.g., ketamine) or an empathogenic agent (e.g., 3,4-Methylenedioxymethamphetamine (MDMA)).

In another aspect, the invention features a method of screening a subject for treatment with a psychedelic agent, the method including: (i) providing a score of one or more predictors of a subject, wherein the score indicates whether a subject is likely to respond to a psychedelic agent; and (ii) based on the score, identifying the subject as likely to have a positive response to the psychedelic agent.

In some embodiments, the subject's response to the psychedelic agent is a positive therapeutic response (e.g., remission).

In some embodiments, the one or more predictors includes one or more trait predictors (e.g., one or more trait predictors including a measure of absorption and/or a measure of mental barriers). In some instances, a high measure of absorption is positively correlated with a positive response. In some instances, a high measure of mental barriers is inversely correlated with a positive response.

In some embodiments, the one or more predictors comprises one or more state predictors (e.g., one or more state predictors selected from the group consisting of a measure of distress, a measure of preoccupation, and a measure of surrender). In some embodiments, a high measure of distress is positively correlated with a positive response. In some embodiments, a high measure of preoccupation is inversely correlated with a positive response. In some embodiments, a high measure of surrender is positively correlated with a positive response. Additionally or alternatively, the one or more predictors may include one or more habits (e.g., frequency of meditation and/or frequency of cannabis use). In some instances, frequency of meditation and/or frequency of cannabis use are positively correlated with a positive therapeutic response.

In some embodiments of any of the preceding methods, the one or more predictors are selected from the group consisting of a measure of absorption, a measure of mental barriers, a measure of meditation frequency, a measure of distress, a measure of preoccupation, a measure of surrender, and a measure of dread.

The score may be a composite score of at least two, at least three, at least four, at least five, at least six, at least seven of the predictors. In some instances, the composite score is a weighted composite score (e.g., wherein each predictor is weighted based on its explanatory power on positive therapeutic response to the psychedelic agent). Explanatory power can be determined by a regression model (e.g., a hierarchical linear multiple regression).

In another aspect, the invention provides a method of screening a subject for treatment with a psychedelic agent, the method including: (i) providing a score of one or more predictors of a subject, wherein the score reflects a high measure of absorption, a high measure of identity distress, and/or a high measure of surrender; (ii) based on the score, identifying the subject as likely to have a positive therapeutic response to the psychedelic agent. In some embodiments, the score further reflects a low measure of mental barriers and/or a low measure of preoccupation.

In some embodiments of any preceding aspect, the positive therapeutic response to the psychedelic agent is preceded by an ME. In some instances, the psychedelic agent is administered as an adjunctive therapy, wherein the subject is being treated with a psychotherapy. In some embodiments of any of the preceding methods, step (ii) further comprises treating the subject with a psychotherapy (e.g., a behavioral psychotherapy, e.g., talk therapy). In some embodiments, the psychotherapy comprises existential therapy. In other embodiments, the psychotherapy comprises self-actualization therapy. In any of the preceding embodiments, step (ii) may occur within a psychotherapeutic setting (e.g., a specialized treatment facility). In some embodiments, the subject is instructed to meditate (e.g., during treatment with a psychedelic agent).

In some embodiments of any of the preceding aspects, the method further includes administering to the subject the psychedelic agent (e.g., a 5-HT_{2A} agonist, e.g., LSD, psilocybin, DOI (±)-1-(2,5-dimethoxyphenyl)-2-aminopropane hydrochloride; (R)-DOI ((R)-1-(2,5-dimethoxy-4-iodophenyI)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine) ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III), or pharmaceutically acceptable salt thereof; or a dissociative agent (e.g., ketamine) or an empathogenic agent (e.g., 3,4-Methylenedioxymethamphetamine (MDMA)).

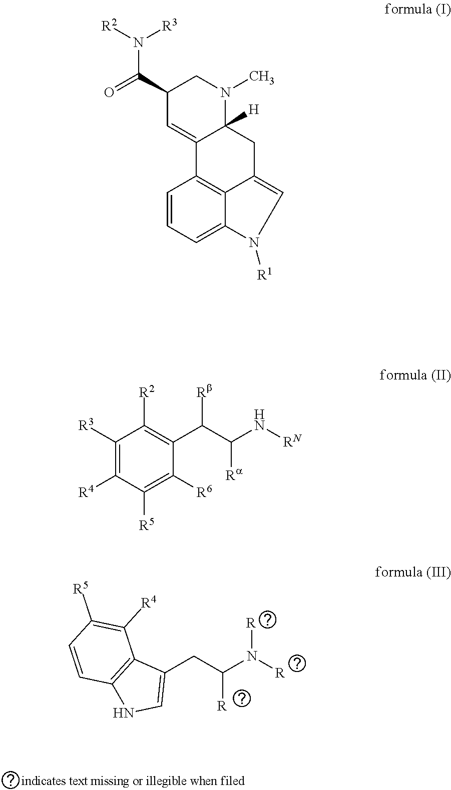

In some embodiments, the score indicates that the subject is unlikely to have a positive response (e.g., unlikely to have a positive therapeutic response or remission). One or more predictors may include one or more trait predictors (e.g., one or more trait predictors including a measure of mental barriers). A high measure of mental barriers may be inversely correlated with a positive response. Additionally or alternatively, the one or more predictors may include one or more state predictors (e.g., one or more state predictors selected from the group consisting of a measure of distress, a measure of preoccupation, and a measure of surrender). In some instances, a high measure of distress is inversely correlated with a positive response. In some instances, a high measure of preoccupation is inversely correlated with a positive response. In some instances, a low measure of surrender is inversely correlated with a positive response. In some embodiments, the one or more predictors are selected from the group consisting of a measure of absorption, a measure of mental barriers, a measure of meditation frequency, a measure of distress, a measure of preoccupation, and a measure of surrender.

In any of the preceding methods, the score may be a composite score of at least two, at least three, at least four, at least five, or at least six of the predictors. The score can be a weighted composite score (e.g., a score weighted based on its explanatory power on positive response to the psychedelic agent). In some embodiments, the explanatory power is determined by a regression model (e.g., a hierarchical linear multiple regression).

In some embodiments of any of the preceding aspects, the subject is likely to have a psychedelic-induced negative experience.

In another aspect, the invention features a method of screening a subject for treatment with a psychedelic agent, the method including: (i) providing a score of one or more predictors of a subject, wherein the score reflects a high measure of mental barriers, a high measure of identity distress, and/or a high measure of preoccupation; and (ii) based on the score, identifying the subject as unlikely to have a positive response to the psychedelic agent. Additionally or alternatively, the score may reflects a low measure of absorption or a low measure of surrender.

In another aspect, the invention features a method of improving mental or physical well-being of a subject, the method including: (i) providing a subject, wherein based on a score of one or more predictors in the subject, the subject has been identified as likely to have a positive therapeutic response to a psychedelic agent (e.g., a 5-HT_{2A} agonist (e.g., lysergic acid diethylamide or psilocybin), a dissociative agent (e.g., ketamine), or an empathogenic agent (e.g., 3,4-Methylenedioxymethamphetamine (MDMA)); and (ii) following step (i), administering to the subject the psychedelic agent. In some embodiments, the method is for treating stress in the subject, treating anxiety in the subject, treating addiction in the subject, treating depression in the subject, or treating a compulsive behavior in the subject. In some embodiments, the subject is obese, and the method comprises promoting weight loss in the subject. In some embodiments, the method is for treating a condition in a subject, improving the mood of a subject, or enhancing the performance of a subject. In some instances, the condition is a psychological disorder.

In some embodiments of the invention, the one or more predictors include one or more trait predictors, such as a measure of absorption and/or a measure of mental barriers. In some instances, a high measure of absorption is positively correlated with a positive therapeutic response. For example, a positive therapeutic response may be predicted if a subject has a high measure of absorption and a high measure of motivations (e.g., a measure of spiritual motivations, norm motivations, pleasure motivations, and/or betterment motivations); a high measure of absorption and a low measure of mental barriers; or a high measure of absorption, a high measure of motivations (e.g., a measure of spiritual motivations, norm motivations, pleasure motivations, and/or betterment motivations), and a low measure of mental barriers. Additionally or alternatively, a high measure of mental barriers is inversely correlated with a positive therapeutic response.

In some embodiments, the one or more predictors include one or more state predictors. In some instances, the one or more state predictors are selected from the group consisting of a measure of distress, a measure of preoccupation, and a measure of motivations (e.g., a measure of spiritual motivations, norm motivations, pleasure motivations, and/or betterment motivations). In some embodiments, a high measure of distress is positively correlated with a positive therapeutic response. For example, a positive therapeutic response may be predicted if a subject has a high measure of distress and a high measure of motivations (e.g., a measure of spiritual motivations, norm motivations, pleasure motivations, and/or betterment motivations). Additionally or alternatively, a high measure of preoccupation may be inversely correlated with a positive therapeutic response. In some embodiments, a high measure of motivations (e.g., a measure of spiritual motivations, norm motivations, pleasure motivations, and/or betterment motivations)is positively correlated with a positive therapeutic response.

In some embodiments, the one or more predictors include one or more habits (e.g., frequency of meditation and/or frequency of cannabis use). In some embodiments, frequency of meditation and/or frequency of cannabis use are positively correlated with a positive therapeutic response.

In some embodiments, the one or more predictors are selected from the group consisting of a measure of absorption, a measure of mental barriers, a measure of meditation frequency, a measure of distress, a measure of preoccupation, a measure of motivations (e.g., a measure of spiritual motivations, norm motivations, pleasure motivations, and/or betterment motivations), and a measure of dread. In some embodiments of any of the preceding methods, the score can be a composite score of at least two, at least three, at least four, at least five, at least six, at least seven of the predictors. The composite score can be a weighted composite score. For example, each predictor can be weighted based on its explanatory power on positive therapeutic response to the psychedelic agent. Explanatory power can be determined, e.g., by a regression model, such as a hierarchical linear multiple regression.

In another aspect, the invention features a method of improving the mental well-being of a subject, the method comprising: (i) providing a subject, wherein the subject has been identified as likely to have a positive therapeutic response to a psychedelic agent based on a high measure of: absorption; identity distress; or motivations (e.g., a measure of spiritual motivations, norm motivations, pleasure motivations, and/or betterment motivations); and (ii) following step (i), administering to the subject the psychedelic agent.

In another aspect, the invention features a method of reducing the risk of developing a psychological disorder in a subject in need thereof, the method comprising: (i) providing a subject, wherein the subject has been identified as likely to have a positive therapeutic response to a psychedelic agent based on a high measure of: absorption; identity distress; or motivations (e.g., a measure of spiritual motivations, norm motivations, pleasure motivations, and/or betterment motivations); and (ii) following step (i), administering to the subject the psychedelic agent.

In some embodiments of any of the preceding aspects, the subject has been identified as likely to have a positive response to a psychedelic agent based on a low measure of: mental barriers; and/or preoccupation.

In some embodiments of any of the preceding methods, the psychedelic agent is administered as an adjunctive therapy, wherein the subject is being treated with, has been treated with, or is going to be treated with a psychotherapy. In some embodiments, step (ii) further comprises treating the subject with a psychotherapy.

The psychotherapy may be a behavioral psychotherapy (e.g., talk therapy). In some instances, the psychotherapy includes existential or humanistic therapy. In some instances, the psychotherapy includes self-actualization therapy. In some embodiments, the subject is instructed to meditate (e.g., during treatment with a psychedelic therapy).

In some embodiments of any of the preceding aspects, step (ii) occurs within a psychotherapeutic setting, such as a specialized treatment facility.

In some embodiments of any of the preceding aspects, the psychological disorder is selected from the group consisting of a depressive disorder, an anxiety disorder, an addiction, or a compulsive behavior disorder. In some instances, the psychological disorder is a depressive disorder (e.g., major depression, melancholic depression, atypical depression, or dysthymia). The depressive disorder may be associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses, any of which may be treated using any of the preceding methods described herein. The psychological disorder may be an anxiety disorder (e.g., end of life anxiety, generalized anxiety disorder, panic disorder, social anxiety, post-traumatic stress disorder, acute stress disorder, obsessive compulsive disorder, or a social phobia). The psychological disorder may be an addiction (substance abuse or an eating disorder). The psychological disorder may be a compulsive disorder (e.g., a primary impulse-control disorder or an obsessive-compulsive disorder). In some embodiments, the methods of the invention can be used to treat a symptom of the psychological disorder (e.g., a psychosomatic symptom or a somatic symptom (e.g., chronic pain, anxiety disproportionate to severity of physical complaints, pain disorder, body dysmorphia, conversion, hysteria, neurological conditions without identifiable cause, or psychosomatic illness)). In some instances, the psychological disorder is a repetitive body-focused behavior (e.g., a tic disorder, e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

In some embodiments, the psychedelic agent is selected from lysergic acid diethylamide, psilocybin, and pharmaceutically acceptable salts thereof. In some embodiments, the psychedelic agent is a 5-HT_{2A} agonist (e.g., LSD, psilocybin, DOI (±)-1-(2,5-dimethoxyphenyl)-2-aminopropane hydrochloride; (R)-DOI ((R)-1-(2,5-dimethoxy-4-iodophenyI)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine); ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III), or a pharmaceutical acceptable salt thereof).

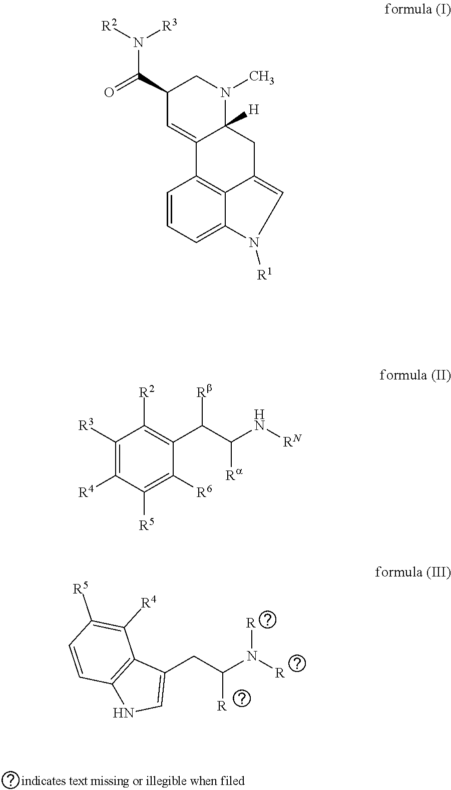

In other embodiments, the psychedelic agent is a dissociative agent (e.g., ketamine) or an empathogenic agent (e.g., 3,4-Methylenedioxymethamphetamine (MDMA)).

In another aspect, the invention features a method of screening a subject for treatment with a psychedelic agent, the method including: (i) providing a score of one or more predictors of a subject, wherein the score indicates whether a subject is likely to respond to a psychedelic agent; and (ii) based on the score, identifying the subject as likely to have a positive response to the psychedelic agent.

In some embodiments, the subject's response to the psychedelic agent is a positive therapeutic response (e.g., remission).

In some embodiments, the one or more predictors includes one or more trait predictors (e.g., one or more trait predictors including a measure of absorption and/or a measure of mental barriers). In some instances, a high measure of absorption is positively correlated with a positive response. In some instances, a high measure of mental barriers is inversely correlated with a positive response.

In some embodiments, the one or more predictors comprises one or more state predictors (e.g., one or more state predictors selected from the group consisting of a measure of distress, a measure of preoccupation, and a measure of motivations (e.g., a measure of spiritual motivations, norm motivations, pleasure motivations, and/or betterment motivations)). In some embodiments, a high measure of distress is positively correlated with a positive response. In some embodiments, a high measure of preoccupation is inversely correlated with a positive response. In some embodiments, a high measure of motivations (e.g., a measure of spiritual motivations, norm motivations, pleasure motivations, and/or betterment motivations) is positively correlated with a positive response. Additionally or alternatively, the one or more predictors may include one or more habits (e.g., frequency of meditation and/or frequency of cannabis use). In some instances, frequency of meditation and/or frequency of cannabis use are positively correlated with a positive therapeutic response.

In some embodiments of any of the preceding methods, the one or more predictors are selected from the group consisting of a measure of absorption, a measure of mental barriers, a measure of meditation frequency, a measure of distress, a measure of preoccupation, a measure of motivations (e.g., a measure of spiritual motivations, norm motivations, pleasure motivations, and/or betterment motivations), and a measure of dread.

The score may be a composite score of at least two, at least three, at least four, at least five, at least six, at least seven of the predictors. In some instances, the composite score is a weighted composite score (e.g., wherein each predictor is weighted based on its explanatory power on positive therapeutic response to the psychedelic agent). Explanatory power can be determined by a regression model (e.g., a hierarchical linear multiple regression).

In another aspect, the invention provides a method of screening a subject for treatment with a psychedelic agent, the method including: (i) providing a score of one or more predictors of a subject, wherein the score reflects a high measure of absorption, a high measure of identity distress, and/or a high measure of motivations (e.g., a measure of spiritual motivations, norm motivations, pleasure motivations, and/or betterment motivations); (ii) based on the score, identifying the subject as likely to have a positive therapeutic response to the psychedelic agent. In some embodiments, the score further reflects a low measure of mental barriers and/or a low measure of preoccupation.

In some embodiments of any preceding aspect, the positive therapeutic response to the psychedelic agent is preceded by an ME. In some instances, the psychedelic agent is administered as an adjunctive therapy, wherein the subject is being treated with a psychotherapy. In some embodiments of any of the preceding methods, step (ii) further comprises treating the subject with a psychotherapy (e.g., a behavioral psychotherapy, e.g., talk therapy). In some embodiments, the psychotherapy comprises existential therapy. In other embodiments, the psychotherapy comprises self-actualization therapy. In any of the preceding embodiments, step (ii) may occur within a psychotherapeutic setting (e.g., a specialized treatment facility). In some embodiments, the subject is instructed to meditate (e.g., during treatment with a psychedelic agent).

In some embodiments of any of the preceding aspects, the method further includes administering to the subject the psychedelic agent (e.g., a 5-HT_{2A} agonist, e.g., LSD, psilocybin, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine) ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III), or pharmaceutically acceptable salt thereof; or a dissociative agent (e.g., ketamine) or an empathogenic agent (e.g., 3,4-Methylenedioxymethamphetamine (MDMA)).

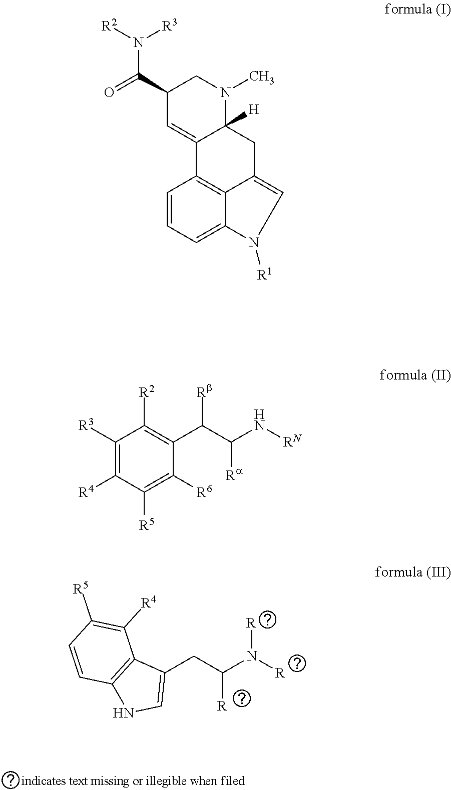

In some embodiments, the score indicates that the subject is unlikely to have a positive response (e.g., unlikely to have a positive therapeutic response or remission). One or more predictors may include one or more trait predictors (e.g., one or more trait predictors including a measure of mental barriers). A high measure of mental barriers may be inversely correlated with a positive response. Additionally or alternatively, the one or more predictors may include one or more state predictors (e.g., one or more state predictors selected from the group consisting of a measure of distress, a measure of preoccupation, and a measure of motivations (e.g., a measure of spiritual motivations, norm motivations, pleasure motivations, and/or betterment motivations)). In some instances, a high measure of distress is inversely correlated with a positive response. In some instances, a high measure of preoccupation is inversely correlated with a positive response. In some instances, a low measure of motivations (e.g., a measure of spiritual motivations, norm motivations, pleasure motivations, and/or betterment motivations) is inversely correlated with a positive response. In some embodiments, the one or more predictors are selected from the group consisting of a measure of absorption, a measure of mental barriers, a measure of meditation frequency, a measure of distress, a measure of preoccupation, and a measure of motivations (e.g., a measure of spiritual motivations, norm motivations, pleasure motivations, and/or betterment motivations).

In any of the preceding methods, the score may be a composite score of at least two, at least three, at least four, at least five, or at least six of the predictors. The score can be a weighted composite score (e.g., a score weighted based on its explanatory power on positive response to the psychedelic agent). In some embodiments, the explanatory power is determined by a regression model (e.g., a hierarchical linear multiple regression).

In some embodiments of any of the preceding aspects, the subject is likely to have a psychedelic-induced negative experience.

In another aspect, the invention features a method of screening a subject for treatment with a psychedelic agent, the method including: (i) providing a score of one or more predictors of a subject, wherein the score reflects a high measure of mental barriers, a high measure of identity distress, and/or a high measure of preoccupation; and (ii) based on the score, identifying the subject as unlikely to have a positive response to the psychedelic agent. Additionally or alternatively, the score may reflects a low measure of absorption or a low measure of motivations (e.g., a measure of spiritual motivations, norm motivations, pleasure motivations, and/or betterment motivations).

In some embodiments of any of the preceding aspects, step (ii) further comprises treating the subject with a psychotherapy (e.g., a behavioral psychotherapy, e.g., talk therapy). In some embodiments, the psychotherapy comprises existential therapy and/or humanistic therapy. In some embodiments, the psychotherapy comprises self-actualization therapy. In some embodiments of any of the preceding aspects, step (ii) occurs within a psychotherapeutic setting (e.g., a specialized treatment facility). In some embodiments, the subject is instructed to meditate (e.g., during treatment with the psychedelic agent).

In some embodiments of any of the preceding aspects, the subject is being screened for treatment to improve his or her physical or mental well-being. The subject may be screened for treatment of stress, treatment of anxiety, treatment of addiction, treatment of depression, or treating of a compulsive behavior. In some embodiments, the subject is obese, and the subject is being screened for treatment for weight loss. In some embodiment, the subject is being screened for treatment for sexual dysfunction. Additionally or alternatively, the subject may be screened for treatment of a condition, improvement of mood, or enhancement of performance. For example, the condition may be a psychological disorder. In some embodiments of any of the preceding aspects, the psychological disorder is selected from the group consisting of a depressive disorder, an anxiety disorder, an addiction, or a compulsive behavior disorder. In some instances, the psychological disorder is a depressive disorder (e.g., major depression, melancholic depression, atypical depression, or dysthymia). The depressive disorder may be associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, decreased initiation of voluntary responses, sexual dysfunction, couples therapy, or relational disorder, any of which may be treated using any of the preceding methods described herein. The psychological disorder may be an anxiety disorder (e.g., end of life anxiety, generalized anxiety disorder, panic disorder, social anxiety, post-traumatic stress disorder, acute stress disorder, obsessive compulsive disorder, or a social phobia). The psychological disorder may be an addiction (substance abuse or an eating disorder). The psychological disorder may be a compulsive disorder (e.g., a primary impulse-control disorder or an obsessive-compulsive disorder). In some embodiments, the methods of the invention can be used to treat a symptom of the psychological disorder (e.g., a psychosomatic symptom or a somatic symptom (e.g., chronic pain, anxiety disproportionate to severity of physical complaints, pain disorder, body dysmorphia, conversion, hysteria, neurological conditions without identifiable cause, or psychosomatic illness)). In some instances, the psychological disorder is a repetitive body-focused behavior (e.g., a tic disorder, e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

As used herein, “well-being” refers to a positive state of health or comfort, e.g., relative to a reference population. As used herein “mental well-being” refers to a positive mental state, relative to a reference population. For example, in an individual having depression, low self-esteem, addiction, compulsion, or anxiety may experience an improvement in mental well-being in response to therapy aimed at improving mood, self-esteem, addiction, compulsion, or anxiety, respectively. As used herein, “physical well-being” refers to one or more positive aspects of an individual's physical health. For example, an improvement of physical well-being includes alleviation of somatic symptoms associated with a psychological disorder, depression, addiction, compulsion, anxiety, or sexual dysfunction. Such symptoms include, for example, chronic pain, pain disorder, relational disorder, body dysmorphia, conversion (e.g., loss of bodily function due to anxiety), hysteria, neurological conditions without identifiable cause, or psychosomatic illness).

As used herein, a “psychological disorder” refers to a condition characterized by a disturbance in one's emotional or behavioral regulation that reflects a dysfunction in the psychological, biological, or developmental processes underlying mental function. Psychological disorders include, but are not limited to depressive disorders (major depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcohol, tobacco, or drug abuse)), and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms. Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, Va.; American Psychiatric Association, 2013).

As used herein, a “trait” or a “trait predictor” is a personality attribute that is consistent over time (e.g., it is not substantially altered by a change in setting). Trait predictors include absorption and mental barriers (i.e., resistance).

As used herein, a “state” or a “state predictor” is an attribute that can be inconsistent over time (e.g., it can be substantially altered by a change in setting). State predictors include distress (e.g., identity distress), preoccupation, surrender, and motivation.

As used herein, a “positive response” or “positive therapeutic response” refers to a measurable clinical benefit with respect to a disorder or a symptom thereof, following treatment with a pharmacological, non-pharmacological, or complex therapy. In some embodiments, a positive therapeutic response is a long-term response (e.g., lasting beyond metabolism and/or excretion of any pharmacological agent), such as remission. A positive therapeutic response may be in comparison to a reference population, as defined below.

As used herein, “absorption” refers to a personality trait characterized by a disposition for situations in which one's total attention fully engages one's representational (i.e., perceptual, enactive, imaginative, and ideational) resources, as defined by Tellegen and Atkinson (Abnormal Psychology 1974, 83(3):268-77). Absorption reflects an individual's cognitive capacity for involvement in sensory and imaginative experiences in ways that alter an individual's perception, memory, and mood with behavioral and biological consequences. Absorption can be quantified using the Tellegen Absorption Scale (TAS), the Multidimensional Personality Questionnaire (MPQ), and variations thereof.

As used herein, “mental barriers” and “cognitive resistance” are used interchangeably to refer to a personality trait characterized by the tendency to reject certain types of information or experiences, as described by Maslow (Journal of Transpersonal Psychology 1970, 2(2):83-90) and James (The varieties of religious experience: A study in human nature, Longmans, Green and Co. New York, N.Y., 1902). The mental barriers can be quantified, e.g., using the six-point mental barriers scale provided in the “Methods” sections of the Examples, below.

As used herein, “distress” and “identity distress” are used interchangeably to refer to the state of turmoil associated with personal change, as described by James (The varieties of religious experience: A study in human nature, Longmans, Green and Co. New York, N.Y., 1902). Identity distress can be quantified using the five-item scale provided by James, or an equivalent variant thereof. For example, in one embodiment, one of the items includes the statements: “I'd no longer had a sense of who I was,” and “I'd felt that my identity was changing.”

As used herein, “preoccupation” refers to the personality state associated with thinking about or being distracted by aspects or events in one's immediate life. Methods to quantify preoccupation, e.g., as reported by a subject during treatment with a psychedelic agent, are provided in the “Methods” section of the Examples, below.

As used herein, “surrender” refers to the personality state associated with acceptance or lack of resistance, as described by James (The varieties of religious experience: A study in human nature, Longmans, Green and Co. New York, N.Y., 1902). Methods to quantify surrender, e.g., as reported by a subject during treatment with a psychedelic agent, are provided in the “Methods” section of the Examples, below.

As used herein, “motivation” or “motivations” refers to the personality state associated with a particular reason for taking a psychedelic agent, such as spiritual motivations, norm motivations, pleasure motivations, and betterment motivations. For example, a subject having spiritual motivations to take a psychedelic agent is more likely to have a psychedelic-induced positive experience. Methods to quantify motivations, e.g., as reported by a subject during treatment with a psychedelic agent, are provided in the “Methods” second of Example 2, below, for example, using the 14 items in the factor loading analysis of Table 6.

As used herein, “dread” refers to one or more negative emotions that may be experienced during treatment with a psychedelic agent (e.g., an abnormally intense feeling of fear, shame, or insignificance). Dread may be categorized as a psychedelic-induced negative experience. Dread can be quantified using the sacred emotions scale set forth by Burdzy (Sacred Emotions Scale (Thesis; 2014), Bowling Green State University, Kentucky).

As used herein, “mystical experience” or “ME” refers to an altered state of consciousness in an individual characterized by at least one of the following key dimensions set forth by Stace (Mysticism and Philosophy, Lippincott, Philadelphia, Pa., 2006): (1) unity, or the sense that all is one; (2) transcendence of time and space; (3) deeply felt positive mood; (4) sense of sacredness, including awe, humility, and reverence; (5) noetic quality, or a feeling of insight with tremendous force of certainty; and (6) alleged ineffability, or an experience that is non-verbal or impossible to describe. An ME can be measured on a continuum scale or can be characterized as a “complete ME,” according to whether or not the degree of experience meets a threshold, according to the methods set forth in Barret et al. (Journal of Psychopharmacology 2015, 29:1182-1190). Characteristics of ME can be self-reported, e.g., using the Mystical Experience Questionnaire (MEQ-43; Griffiths et al., Psychopharmacology 2006, 187:268-283) or equivalent variant thereof (e.g., MEQ-30; MacLean et al, Journal for the Scientific Study of Religion 2012, 51:721-737; Barret et al., Journal of Psychopharmacology 2015, 29:1182-1190). ME can be approximated using a correlate. For example, the “ocean boundlessness” dimension of the 5D-ASC provided by Studerus et al. (PLoS ONE 2012, 7) can be used as a correlate measure of ME.

As used herein, a “measure” of a predictor (e.g., a measure of absorption, a measure of mental barriers, a measure of distress, a measure of preoccupation, or a measure of surrender) refers to a metric derived from a readout that is descriptive of the predictor. For example, a measure of absorption can be a TAS score or a derivative thereof (e.g., a TAS score weighted by a coefficient).

As used herein, a “correlate” of a predictor refers to any attribute that correlates with the predictor to which refers, as determined by a correlation coefficient. In some embodiments, the correlation coefficient is r≥0.10 (e.g., r≥0.15, r≥0.20, r≥0.25, r≥0.30, r≥0.35, r≥0.40, r≥0.50, r≥0.60, r≥0.70, r≥0.80, r≥0.90, or r≥0.95). In some embodiments, the correlation coefficient is r≥0.20. A correlation coefficient can be derived from a correlation matrix, such as that provided in Tables 1 and 10.

As used herein, a subject is said to have a “high measure” of a predictor if the measure of the predictor is above a threshold (e.g., a predetermined threshold on an established scale or a threshold set by cumulative results of a reference population). For example, a measure of a predictor may be high if it is in the top 90^{th} percentile among a reference population (e.g., in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Alternatively, a measure of a predictor of a subject may be high if the subject scores an average (e.g., mean, median, or mode) of ≥0.5, ≥1, ≥1.5, ≥2, ≥2.5, ≥3, or ≥3.5 on a 4-point Likert scale; an average (e.g., mean, median, or mode) of ≥0.5, ≥1, ≥1.5, ≥2, ≥2.5, ≥3, ≥3.5, ≥4.0, ≥4.5, ≥5.0, or ≥5.5 on a 6-point Likert scale; or an average (e.g., mean, median, or mode) anywhere in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile of a Likert scale having any number of items.

Conversely, as used herein, a subject is said to have a “low measure” of a predictor if the measure of the predictor is below a threshold (e.g., a predetermined threshold on an established scale or a threshold set by cumulative results of a reference population). For example, a measure of a predictor may be low if it is in the bottom 90^{th} percentile among a reference population (e.g., in the bottom 80^{th} percentile, the bottom 70^{th} percentile, the bottom 60^{th} percentile, the bottom 50^{th} percentile, the bottom 40^{th} percentile, the bottom 30^{th} percentile, the bottom 25^{th} percentile, the bottom 20^{th} percentile, the bottom 15^{th} percentile, the bottom 10^{th} percentile, or the bottom 5^{th} percentile among a reference population). Alternatively, a measure of a predictor of a subject may be low if the subject scores an average (e.g., mean, median, or mode) of ≥3.5, ≥3, ≥2.5, ≥2, ≥1.5, ≥1, ≥0.5, or 0 on a 4-point Likert scale; an average (e.g., mean, median, or mode) of ≥5.5, ≥5.0, ≥4.5, ≥4.0, ≥3.5, ≥3, ≥2.5, ≥2, ≥1.5, ≥1, ≥0.5, or 0 on a 6-point Likert scale; or an average (e.g., mean, median, or mode) anywhere in the bottom 80^{th} percentile, the bottom 70^{th} percentile, the bottom 60^{th} percentile, the bottom 50^{th} percentile, the bottom 40^{th} percentile, the bottom 30^{th} percentile, the bottom 25^{th} percentile, the bottom 20^{th} percentile, the bottom 15^{th} percentile, the bottom 10^{th} percentile, or the bottom 5^{th} percentile of a Likert scale having any number of items.

As used herein, a “reference population” refers to a group of individuals to whom a subject's one or more attributes, predictors, or responses is compared. A reference population may refer to the entirety of a sample group that have participated in or completed an assessment (e.g., from previously gathered normative data). In some cases, outliers or non-compliant individuals may be removed from the results of assessment, in which case, those individuals are excluded from the reference population. The reference population may be a group of subjects who have been assessed for likelihood of responding to a psychedelic treatment, a group of subjects who have a psychological disorder (e.g., any of the psychological disorders described herein), a group of individuals who have had experience with a psychedelic agent, any combination thereof, or a group of individuals arbitrarily chosen from the public or from a particular demographic.

For a given population of subjects that are identified as “likely” to have a particular response to a treatment (e.g., a positive therapeutic response to a treatment), the frequency of subjects who have the response is at least 10% greater than the frequency of subjects who have the response within a reference population. For example, in a population of subjects being treated for depression with a psychedelic agent who have been screened and identified as likely to have a positive therapeutic response, 50% of the subjects may have a positive therapeutic response, whereas in a population of subjects who undergo the treatment without having been screened, the treatment might result in only 35% responsiveness. Hence, each of the subjects of the first group is referred to as “likely to have a positive therapeutic response.” In some embodiments, the frequency of subjects who have the response is at least 15% (e.g., at least 20%, at least 25%, at least 30%, at least 35%, at least 40%, at least 45%, at least 50%, at least 60%, at least 70%, at least 80% or more) greater than the frequency of subjects who have the response within a reference population.

Similarly, for a given population of subjects that are identified as “unlikely” to have a particular response to a treatment (e.g., a positive therapeutic response to a treatment), the frequency of subjects who have the response is at least 10% less than the frequency of subjects who have the response within a reference population. For example, in a population of subjects being treated for depression with a psychedelic agent who have been screened and identified as unlikely to have a positive therapeutic response, 20% of the subjects may have a positive therapeutic response. In a population of subjects who undergo the treatment without having been screened, the treatment might result in 35% responsiveness. Hence, each of the subjects of the first group is referred to as “unlikely to have a positive therapeutic response.” In some embodiments, the frequency of subjects who have the response is at least 15% (e.g., at least 20%, at least 25%, at least 30%, at least 35%, at least 40%, at least 45%, at least 50%, at least 60%, at least 70%, or at least 80%) less than the frequency of subjects who have the response within a reference population.

As used herein, an “equivalent variant” refers to a means of assessing an individual that yields an equivalent, near equivalent, or superior statistical result (e.g., explanatory power or correlation coefficient) relative to a reference assessment means. For example, equivalent variants of the TAS that have been adapted for various purposes are known in the art and ultimately yield a measure that is equivalent, near equivalent, or superior in describing the degree of a subject's absorption.

As used herein, a “psychedelic agent” refers to a compound capable of inducing an altered state of consciousness, i.e., a marked deviation in the subjective experience or psychological functioning of a normal individual from his or her usual waking consciousness. Altered states of consciousness can be monitored, evaluated, and/or quantified using any of a variety of methods known in the art including, without limitation, Dittrich's APZ (Abnormal Mental States) questionnaire, and its revised versions, OAV and 5D-ASC (see, for example, Dittrich et al., A Pharmacopsychiatry 1998, 31:80; Studerus et al., PLoS ONE 2010, 5). Psychedelic agents include 5-HT_{2A} agonists (e.g., lysergic acid diethylamide (LSD), empathogenic agents (i.e., serotonin (5-HT) releasing agents; e.g., 3,4-methylenedioxymethamphetamine (MDMA)), and dissociative agents (i.e., N-Methyl-D-aspartate (NMDA) receptor agonists; e.g., ketamine).

As used herein, a “5-HT_{2A} agonist” refers to a compound that increases the activity of a 5-hydroxytryptamine 2A receptor. Examples of such agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III).

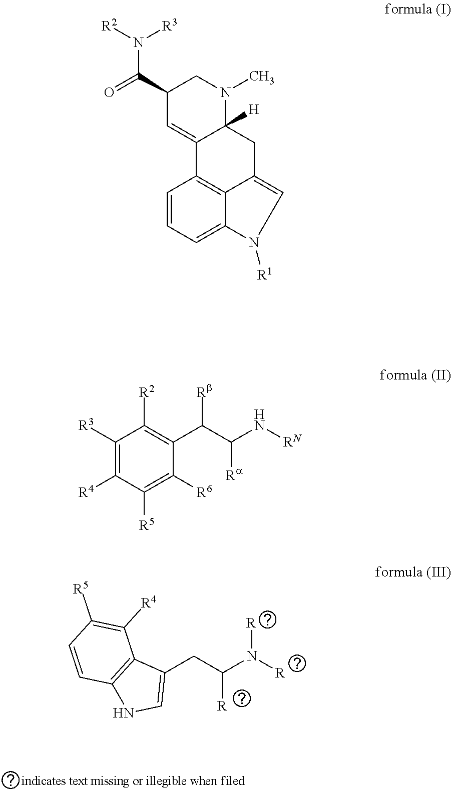

As used herein, the term “treating” refers to administering a pharmaceutical composition for therapeutic purposes. To “treat a disorder” or use for “therapeutic treatment” refers to administering treatment to a patient already suffering from a disease to ameliorate the disease or one or more symptoms thereof to improve the patient's condition. The methods of the invention can also be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

The term “administration” or “administering” refers to a method of giving a dosage of a pharmaceutical composition to a subject, where the method is, e.g., oral, topical, transdermal, by inhalation, intravenous, intraperitoneal, intracerebroventricular, intrathecal, or intramuscular.

As used herein, a “psychotherapy” refers to a non-pharmaceutical therapy in which the subject is psychologically engaged, directly or indirectly (e.g., by dialogue), in an effort to restore a normal psychological condition; to reduce the risk of developing a psychological condition, disorder, or one or more symptoms thereof; and/or to alleviate a psychological condition, disorder, or one or more symptoms thereof. Psychotherapy includes Behavioral Activation (BA), Cognitive Behavioral Therapy (CBT), Interpersonal psychotherapy (IPT), Psychoanalysis, Hypnotherapy, Psychedelic Psychotherapy, Psycholytic Psychotherapy, and other therapies. In some embodiments, a subject undergoes psychotherapy in conjunction with (e.g., prior to, during, and/or after) a pharmaceutical therapy, such as a psychedelic therapy.

As used herein, a “psychotherapeutic setting” refers to an environment configured to be substantially free of distraction or stress to facilitate a positive therapeutic response in a subject.

As used herein, a “specialized treatment facility” refers to a particular psychotherapeutic setting in which subjects are evaluated for treatment by a complex therapy (i.e., a therapy including both pharmaceutical (e.g., psychedelic) and non-pharmaceutical (e.g., psychotherapy) treatments).

Other features and advantages of the invention will be apparent from the following Detailed Description, Examples, Figure, and Claims.

### BRIEF DESCRIPTION OF THE FIGURES

FIG. 1 is a bar graph showing the accuracy of the predictive model of Example 1.

FIG. 2 is a path diagram of the mediation role of mystical-OBN in predicting positive change.

### DETAILED DESCRIPTION OF THE INVENTION

Psychedelic agents can induce long-term positive responses (e.g., improvement in mental or physical well-being or alleviation of symptoms of a psychological disorder) in a subject susceptible thereto, and, conversely, certain attributes may render some subjects less likely to have a positive response and more likely to experience a psychedelic-induced negative experience (e.g., an experience of dread).

The present invention is based, at least in part, on the results of a systematic study quantifying the relative significance of various predictors (e.g., trait and state predictors) on a subject's susceptibility to the positive responses or negative experiences (e.g., dread) associated with psychedelic treatment. This disclosure enables methods of determining whether a subject is likely to have a positive response to a psychedelic agent by assessing and weighing specific predictors. Thus, methods described herein provide inclusion or exclusion criteria for indicating whether a subject suffering from a psychological disorder is likely to respond to treatment with a psychedelic agent (e.g., lysergic acid diethylamide or psilocybin). Methods of the invention also include methods of administering a psychedelic agent to a subject in need thereof, on the basis of the subject's likelihood of responding positively to the treatment, based on the screening methods provided herein.

### Prediction of Positive Response to Psychedelic Therapy

In subjects in need of psychedelic therapy (e.g., subjects in need of improved mental or physical well-being, e.g., subjects having a psychological disorder) a long-term, transformational life change (e.g., remission) can be accompanied, facilitated, or enhanced by a mystical experience (ME) induced by ingestion of a psychedelic agent.
Trait Predictors of Positive Response to Psychedelic Therapy

In some embodiments of the invention, predictors of positive response include trait predictors, such as absorption and mental barriers. High measures of absorption may indicate that a subject is likely to have a positive response to psychedelic treatment, while low measures of mental barriers may indicate that a subject is likely to have a positive response to psychedelic treatment.

A subject can be identified as likely to have a positive response to psychedelic treatment if the subject's measure of absorption (e.g., as measured by the Tellegen Absorption Scale or variant thereof) is determined to be in the top 90^{th} percentile among a reference population (e.g., the subject's measure of absorption is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as likely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of absorption. In some embodiments, the subject is identified as likely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of absorption.

In some instances, a subject is identified as likely to have a positive response to psychedelic treatment if the subject's measure of mental barriers (e.g., resistance, e.g., cognitive resistance) is determined to be in the bottom 90^{th} percentile among a reference population (e.g., the subject's measure of absorption is determined to be in the bottom 80^{th} percentile, the bottom 70^{th} percentile, the bottom 60^{th} percentile, the bottom 50^{th} percentile, the bottom 40^{th} percentile, the bottom 30^{th} percentile, the bottom 25^{th} percentile, the bottom 20^{th} percentile, the bottom 15^{th} percentile, the bottom 10^{th} percentile, or the bottom 5^{th} percentile among a reference population). Additionally or alternatively, a subject can be identified as likely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of mental barriers. In some embodiments, the subject is identified as likely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of mental barriers.

A subject's measure of mental barriers can also be quantified using a scale based on the degree to which a subject agrees with the following six statements or equivalents thereof:

(i) “I reject ideas that can't be logically explained;”

(ii) “I reject ideas that are not supported by experts in the field;”

(iii) “I reject ideas that are based solely on others' personal experiences;”

(iv) “I only accept ideas that have scientific evidence behind them;”

(v) “I have had experiences of knowing something without knowing how I knew it;” and

(vi) “My intuition has helped me at times;”
wherein statements (i)-(iv) are included in a first component, and statements (v) and (vi) are included in a second component of a principle component analysis. In some embodiments, Varimax rotated loadings range from 0.755 to 0.835 for the component including statements (i)-(iv). In some embodiments, Varimax rotated loadings range from −0.864 to −0.872 for the component including statements (v) and (vi).
State Predictors of Positive Response to Psychedelic Therapy

Predictors of positive response may additionally or alternatively include state predictors, such as distress, preoccupation, surrender, and motivations (e.g., spiritual motivations, norm motivations, pleasure motivations, or betterment motivations). High measures of distress in combination with high measures of surrender may indicate that a subject is likely to have a positive response to psychedelic treatment, while low measures of preoccupation may indicate that a subject is likely to have a positive response to psychedelic treatment.

A subject can be identified as likely to have a positive response to psychedelic treatment if the subject's measure of identity distress (e.g., as measured according to James (The varieties of religious experience: A study in human nature, Longmans, Green and Co. New York, N.Y., 1902)) is determined to be in the top 90^{th} percentile among a reference population (e.g., the subject's measure of identity distress is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as likely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of identity distress. In some embodiments, the subject is identified as likely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of identity distress.

To assess the effect of a subject's state on his or her likelihood of having a particular response to a psychedelic therapy, an assessment may consider a period of up to several days, weeks, months, and/or years prior to the assessment.
Psychedelic-Induced Mystical Experience as a Predictor of Positive Therapeutic Response

In some embodiments of the invention, a subject may have a psychedelic-induced ME. The occurrence of an ME in a subject suggests that the subject may be more likely to have a positive therapeutic response to the psychedelic treatment (e.g., remission). Methods of determining whether a subject has had an ME are known in the art and provided herein.

### Prediction of Non-Responses to Psychedelic Therapy

In subjects in need of improved mental or physical well-being (e.g., subjects having a psychological disorder), ingestion of a psychedelic agent may not be the most effective treatment (e.g., may not lead to a positive therapeutic response) and may, for example, lead to negative experiences, such as dread. Accordingly, methods of the invention allow such subjects to be identified and, in some cases, excluded form a psychedelic treatment regimen. Rather, subjects unlikely to have a positive response to a psychedelic agent and/or subjects likely to experience psychedelic-induced dread may be treated with non-pharmacological means, such as psychotherapy (e.g., behavioral psychotherapy).
Trait Predictors of Non-Responses to Psychedelic Therapy

In some embodiments of the invention, predictors of non-responses or psychedelic-induced negative experiences (e.g., dread) include trait predictors, such as mental barriers. High measures of mental barriers may indicate that a subject is unlikely to have a positive response to psychedelic treatment. In some instances, a high measure of mental barriers indicates that a subject is more likely to have a psychedelic-induced negative experience (e.g., dread).

In some instances, a subject is identified as unlikely to have a positive response to psychedelic treatment if the subject's measure of mental barriers (e.g., resistance, e.g., cognitive resistance) is determined to be in the top 90^{th} percentile among a reference population (e.g., the subject's measure of mental barriers is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as unlikely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of mental barriers. In some embodiments, the subject is identified as unlikely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of mental barriers.

In some embodiments, a subject is identified as likely to have a psychedelic-induced negative experience (e.g., dread) in response to psychedelic treatment if the subject's measure of mental barriers (e.g., resistance, e.g., cognitive resistance) is determined to be in the top 90^{th} percentile among a reference population (e.g., the subject's measure of mental barriers is determined to be in the top 80th percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as likely to have a psychedelic-induced negative experience (e.g., dread) in response to psychedelic treatment if the subject scores an average (e.g., mean, median, or mode) of ≥3 on a 4-point Likert scale or a ≥4 on a 6-point Likert scale, wherein the Likert scale is configured to determine the subject's measure of mental barriers. In some embodiments, the subject is identified as likely to have a psychedelic-induced negative experience (e.g., dread) in response to psychedelic treatment if the subject scores an average (e.g., mean, median, or mode) of ≥2 on a 4-point Likert scale or ≥3 on a 6-point Likert scale, wherein the Likert scale is configured to determine the subject's measure of mental barriers.

A subject's measure of mental barriers can also be quantified using a scale based on the degree to which a subject agrees with the following six statements or equivalents thereof:

(i) “I reject ideas that can't be logically explained;”

(ii) “I reject ideas that are not supported by experts in the field;”

(iii) “I reject ideas that are based solely on others' personal experiences;”

(iv) “I only accept ideas that have scientific evidence behind them;”

(v) “I have had experiences of knowing something without knowing how I knew it;” and

(vi) “My intuition has helped me at times;”
wherein statements (i)-(iv) are included in a first component, and statements (v) and (vi) are included in a second component of a principle component analysis. In some embodiments, Varimax rotated loadings range from 0.755 to 0.835 for the component including statements (i)-(iv). In some embodiments, Varimax rotated loadings range from −0.864 to −0.872 for the component including statements (v) and (vi).
State Predictors of Non-Responses to Psychedelic Therapy

Predictors of non-responses (e.g., no clinical improvement) may additionally or alternatively include state predictors, such as distress, preoccupation, and surrender. High measures of distress and/or preoccupation may indicate that a subject is unlikely to have a positive response to psychedelic treatment and may indicate that the subject is likely to have a psychedelic-induced negative experience. For example, in some embodiments, a high measure of distress and a high measure of preoccupation indicates that a subject is unlikely to have a positive response to psychedelic treatment.

In some embodiments, a subject can be identified as unlikely to have a positive response to psychedelic treatment if the subject's measure of identity distress (e.g., as measured according to James (The varieties of religious experience: A study in human nature, Longmans, Green and Co. New York, N.Y., 1902)) is determined to be in the top 90^{th} percentile among a reference population (e.g., the subject's measure of identity distress is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as unlikely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of identity distress. In some embodiments, the subject is identified as unlikely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of identity distress.

In some embodiments, a subject can be identified as likely to have a psychedelic-induced negative experience (e.g., dread) in response to psychedelic treatment if the subject's measure of identity distress (e.g., as measured according to James (The varieties of religious experience: A study in human nature, Longmans, Green and Co. New York, N.Y., 1902)) is determined to be in the top 90^{th} percentile among a reference population (e.g., the subject's measure of identity distress is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as likely to have a psychedelic-induced negative experience (e.g., dread) in response to psychedelic treatment if the subject scores an average (e.g., mean, median, or mode) of ≥2 on a 4-point Likert scale or ≥3 on a 6-point Likert scale, wherein the Likert scale is configured to determine the subject's measure of identity distress. In some embodiments, the subject is identified as likely to have a psychedelic-induced negative experience (e.g., dread) in response to psychedelic treatment if the subject scores an average (e.g., mean, median, or mode) of ≥3 on a 4-point Likert scale or ≥4 on a 6-point Likert scale, wherein the Likert scale is configured to determine the subject's measure of identity distress.

In some embodiments, a subject can be identified as unlikely to have a positive response to psychedelic treatment if the subject's measure of preoccupation is determined to be in the top 90th percentile among a reference population (e.g., the subject's measure of preoccupation is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as unlikely to have a positive response to psychedelic treatment if the subject scores an average (e.g., mean, median, or mode) of ≥2 on a 4-point Likert scale or ≥3 on a 6-point Likert scale, wherein the Likert scale is configured to determine the subject's measure of preoccupation. In some embodiments, the subject is identified as unlikely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of preoccupation.

In some embodiments, a subject can be identified as likely to have a psychedelic-induced negative experience (e.g., dread) in response to psychedelic treatment if the subject's measure of preoccupation is determined to be in the top 90^{th} percentile among a reference population (e.g., the subject's measure of preoccupation is determined to be in the top 80^{th} percentile, the top 70^{th} percentile, the top 60^{th} percentile, the top 50^{th} percentile, the top 40^{th} percentile, the top 30^{th} percentile, the top 25^{th} percentile, the top 20^{th} percentile, the top 15^{th} percentile, the top 10^{th} percentile, or the top 5^{th} percentile among a reference population). Additionally or alternatively, a subject can be identified as likely to have a psychedelic-induced negative experience (e.g., dread) in response to psychedelic treatment if the subject scores an average (e.g., mean, median, or mode) of ≥2 on a 4-point Likert scale or ≥3 on a 6-point Likert scale, wherein the Likert scale is configured to determine the subject's measure of preoccupation. In some embodiments, the subject is identified as unlikely to have a positive response to psychedelic treatment if the subject scores an average (e.g., mean, median, or mode) of ≥3 on a 4-point Likert scale or ≥4 on a 6-point Likert scale, wherein the Likert scale is configured to determine the subject's measure of preoccupation.

### Deriving Scores from Multiple Predictors

A subject can be identified (e.g., as likely to have a positive response to a psychedelic treatment; as unlikely to have a positive response to a psychedelic treatment; or as likely to have a psychedelic-induced negative experience) based on a composite score (e.g., a composite of multiple predictors). In some instances, a composite score reflects predictors that are weighted. In some embodiments, the predictors are weighted based, wholly or partially, on their relative explanatory power on a dependent variable (e.g., their relative explanatory power on the type of response to a psychedelic treatment, e.g., their relative explanatory power on ME, dread, and/or long-term positive response, e.g., remission). Explanatory power of any predictor described herein can be calculated using methods known in the art (e.g., using a regression model, e.g., hierarchical linear multiple regression; structural equation modeling; or path analysis) and described, e.g., in the “Statistical Approach” section of the Examples, below (R² values in Tables 2-4 and 11-14 represent explanatory power).

In some instances, a composite score reflects predictors that are weighted based, wholly or partially, on a correlation between the predictors and the dependent variable (e.g., on the correlation between the predictor and the type of response to a psychedelic treatment, e.g., their explanatory power on ME, dread, and/or long-term positive response, e.g., remission). Any known means to derive of a correlation (e.g., given by a correlation coefficient) are suitable for use as part of the methods described herein. Tables 1 and 10 provide exemplary correlation matrices between attributes and dependent measures.

In some instances, a single attribute may be a predictor of positive response or a predictor of non-response, depending on a measure of another one or more predictors. For example, a high measure of identity distress may indicate that a subject is likely to have a positive response to a psychedelic treatment if the subject also has a high measure of surrender. Conversely, if the subject has a high measure of identity distress and a low measure of surrender or a high measure of preoccupation, the subject may be identified as unlikely to have a positive response to the psychedelic therapy and may be more likely to have a psychedelic-induced negative experience. Such complex predictor relationships are described in detail in Examples 1-3.

### Therapies

The methods of the invention can be used to screen for subjects that are likely to have a positive therapeutic response to psychedelic treatments for improving mental or physical well-being. Improving mental well-being includes treating or preventing anxiety disorders (e.g., end of life anxiety, generalized anxiety disorder, panic disorder, social anxiety, post-traumatic stress disorder, acute stress disorder, obsessive compulsive disorder, and social phobias) or stable depressive disorders (e.g., major depression, melancholic depression, atypical depression, or dysthymia).
Treatment Facilities

In some embodiments, the methods of treatment and screening provided herein are performed in the context of an authorized treatment facility (e.g., a specialized treatment facility) configured to provide complex therapies to subjects in need thereof. Complex therapies may involve both pharmaceutical (e.g., psychedelic agent-based) and non-pharmaceutical treatments designed according to a subject's specific needs. For example, methods provided herein enable a practitioner to determine whether a subject is likely to benefit from a psychedelic treatment and act accordingly. In many instances, a psychedelic treatment regimen may not be prescribed (and may be detrimental) outside of the context of a specialized treatment facility in which a subject has access to adjunctive psychotherapy (e.g., behavioral therapy, existential, humanistic, or self-actualization therapy). Specialized treatment facilities can be configured to enhance the safety and efficacy of therapy (e.g., psychedelic therapy and/or complex therapy) through control and use of audio, visual, and other environmental factors. In general, specialized treatment facilities feature a staff that has training and expertise in administering and overseeing psychedelic therapy, psychotherapy, and/or complex therapy.

Treatment facilities in which psychedelic and/or complex therapies can be administered include other settings that are authorized to administer therapies including psychedelic therapies, adjunctive psychotherapies, and/or complex therapies. For example, authorized treatment facilities may be associated with a hospital, a mental health clinic, or a retreat center. Treatment facilities may be in-patient or out-patient facilities and may provide screening, evaluation, and follow-up services. In some embodiments, treatment facilities may be associated with a research facility/program.
Addictions and Substance Abuse

The methods and compositions of the invention can be used to treat substance abuse, drug addictions, and addictive behaviors. Addictive behaviors which can be treated using the methods of the invention include food addiction, binge eating disorder, pathological gambling, pathological use of electronic devices, pathological use of electronic video games, pathological use of electronic communication devices, pathological use of cellular telephones, addiction to pornography, sex addiction, obsessive-compulsive disorder, compulsive spending, intermittent explosive disorder, kleptomania, pyromania, trichotillomania, compulsive over-exercising, and compulsive overworking. Drug addictions which can be treated using the methods of the invention include addictions to recreational drugs, as well as addictive medications. Examples of addictive agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine.
Anxiety Disorders

The methods of the invention can be used to treat anxiety disorders. Anxiety is broadly defined as a state of unwarranted or inappropriate worry often accompanied by restlessness, tension, distraction, irritability and sleep disturbances. This disproportionate response to environmental stimuli can hyperactivate the hypothalamic-pituitary-adrenal axis and the autonomic nervous system, resulting in somatic manifestation of anxiety, including shortness of breath, sweating, nausea, rapid heartbeat and elevated blood pressure (Sanford et al., Pharmacol. Ther. 88:197 (2000)). Anxiety disorders represent a range of conditions and as a result have been classified into multiple distinct conditions, including generalized anxiety disorder (GAD), panic disorder, social anxiety, post-traumatic stress disorder (PTSD), acute stress disorder (ASD), obsessive compulsive disorder (OCD), and social phobias (Sanford et al., Acta. Psychiatr. Scand. 1998, Suppl. 393:74).

Generalized anxiety disorder (GAD) is the most commonly occurring of the anxiety disorders and is characterized by excessive and persistent worries. In the general population the lifetime prevalence rate of GAD ranges from 4.1 to 6.6% with somewhat higher rates in women than in men. The individual with GAD worries about life events such as marital relationships, job performance, health, money, and social status. Individuals with GAD startle easily and may suffer from depression. Some of the specific symptoms of GAD include restlessness, motor tension, difficulty concentrating, and irritability. The severity of the symptoms over time may be linked to the changing nature of the environmental stressor. With increasing age, GAD symptoms become less severe.

Panic disorder is a well-studied psychiatric condition that consists of multiple disabling panic attacks characterized by an intense autonomic arousal. In addition, heightened fear and anxiety states occur both during and between panic attacks. Approximately 3% of women and 1.5% of men have panic attacks. During a panic attack, the individual experiences multiple symptoms including light-headedness, a pounding heart and difficulty in breathing.

Post-traumatic stress disorder (PTSD) is a disorder characterized by intense fear and anxiety states that require psychiatric treatment. PTSD often results from exposure to a life threatening or traumatic event. Individuals with PTSD can have recurring thoughts of the terrifying event. Reenactment of the event varies in duration from a few seconds or hours to several days.
Psychedelic Agents

The invention features methods of screening a subject for candidacy for treatment with a psychedelic agent, as well as methods of treatment including administering a psychedelic agent. A psychedelic agent useful as part of the invention is a compound capable of inducing an altered state of consciousness, i.e., a marked deviation in the subjective experience or psychological functioning of a normal individual from his or her usual waking consciousness. Psychedelic agents include 5-HT_{2A} agonists (e.g., lysergic acid diethylamide (LSD), empathogenic agents (i.e., serotonin (5-HT) releasing agents; e.g., 3,4-methylenedioxymethamphetamine (MDMA)), and dissociative agents (i.e., N-Methyl-D-aspartate (NMDA) receptor agonists; e.g., ketamine).

5-HT_{2A} agonists include psilocybin, LSD, DOI (±)-1-(2,5-dimethoxyphenyI)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); ibogaine; a compound of formula (I); a compound of formula (II); or a compound of formula (III).

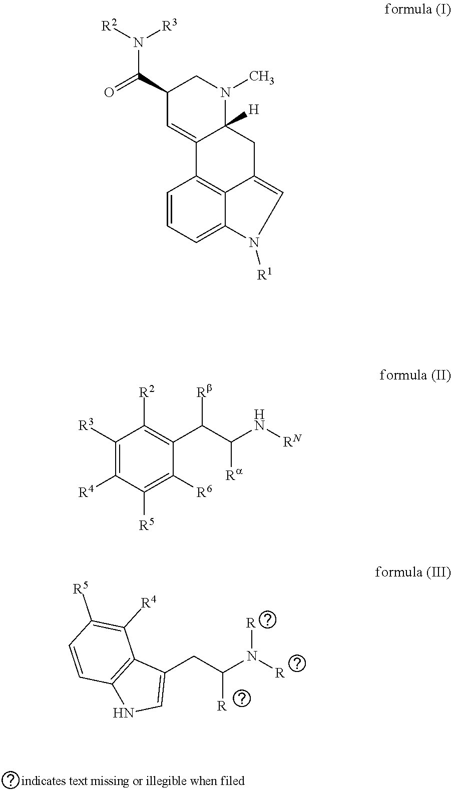

Solid Dosage Forms for Oral Use

Formulations of psychedelic agents for oral use include tablets containing the psychedelic agent in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the psychedelic drug substance in a predetermined pattern (e.g., in order to achieve a controlled release formulation) or it may be adapted not to release the psychedelic drug substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the psychedelic drug substance). The coating may be applied on the solid dosage form in a similar manner as that described in Encyclopedia of Pharmaceutical Technology, supra.

Formulations for oral use may also be presented as chewable tablets, or as hard gelatin capsules wherein the psychedelic compound is mixed with an inert solid diluent (e.g., potato starch, lactose, microcrystalline cellulose, calcium carbonate, calcium phosphate or kaolin), or as soft gelatin capsules wherein the psychedelic compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.
Liquids for Oral Administration

Powders, dispersible powders, or granules suitable for preparation of an aqueous suspension by addition of water are convenient dosage forms for oral administration of psychedelic agents. Formulation as a suspension provides the psychedelic agent in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.
Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Hayes (Remington: The Science and Practice of Pharmacy, volume I and volume II. Twenty-second edition. Philadelphia, 2012).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the psychedelic compound, the composition may include suitable parenterally acceptable carriers and/or excipients. The psychedelic drug may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the psychedelic drug is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### EXAMPLES

### Example 1. Predictors of Psychedelic-Induced Mystical Experience and Positive Response

The following study developed and characterized a set of mental state and trait predictors of mystical experience (ME) to identify predictors of positive response to psilocybin treatment.

### Methods

A retrospective survey study was conducted based on crowdsourced online data gathered from individuals who had volitionally consumed psilocybin within the past year. Data were collected about participants' traits and demographic background, and then individuals were primed to recall their life experience before, at the time of ingestion, during, and after the psilocybin before completing scales to measure hypothesized traits and states. Previously validated scales were used where available (e.g., Tellegen Absorption Scale, MEQ30), and new scales were developed and tested for traits and states without existing measures (mental barriers, identity distress, state of surrender). A hierarchical regression model was used to test the relationships, and a path analysis was conducted to test the mediating capacity of ME.

This study was approved by the Institutional Review Board at Dickinson State University (IRB # DSU201604), and all data were collected in accordance with the stipulations therein. Participants read an informed consent document and agreed to the conditions prior to receiving the password for the survey. All existing scales were used with written permission.
Survey

The online survey consisted of 276 items organized into seven pages on an online survey tool.

Participants were directed to the relevant webpage after reading informed consent text, completing screening and agreeing to participate in accordance with the stipulations of the Institutional Review Board. Scaled items in all but the long-term outcomes used 4-point Likert scales ranging from 1=Strongly Disagree to 4=Strongly Agree. Answers were required for each item, but each included an option “I prefer not to answer.”

The survey was organized into six sections: (1) Background, (2) Traits, (3) Days and weeks before the experience, (4) Onset of experience, (5) During experience, and (6) After experience. The survey was organized with questions to activate memories of each aspect of participants' psychedelic experience before completing the scale items. On the last page of the survey, participants were asked to indicate on a four-point scale how carefully and accurately they had been able to respond to the questions, and were told that answers would not affect financial compensation of $1.00.
Background Items

Background items requested information about participants' sex, education, age, and importance of formal religion and spirituality in their childhood family and in their lives today. Participants were also asked about the date of the psilocybin experience they were describing and the extent to which they had prior experience with psychedelic drugs.
Trait Measures

Items from a variety of trait scales were intermixed randomly and presented in five-statement blocks, along with four attention items written to be semantically similar to the surrounding items (e.g., I will mark four for this item”).

These scales included:

Tellegen Absorption Scale (TAS)

The TAS is a 34-item trait or a disposition “for having episodes of “total” attention that fully engage one's representational (i.e., perceptual, enactive, imaginative, and ideational) resources” developed to predict hypnotizability and previously found to also predict ME-related phenomena in psychedelic sessions (Studerus et al., PLoS ONE 2012, 7).

Rational Emotions Inventory (REI).

The REI consists of ten items to measure tendencies for rational versus emotional thought processes (Epstein et al, Journal of Personality and Social Pathology 1996, 71:390-405).

Mental Barriers Scale

This is a newly developed six-item scale including six items addressing the tendency to reject certain types of information in conjunction with descriptions by James (The varieties of religious experience: A study in human nature, Longmans, Green and Co. New York, N.Y., 1902) and Abraham Maslow (Journal of Transpersonal Psychology 1970, 2(2):83-90). Sample items include “I reject ideas that cannot be scientifically proven” and “If it doesn't make logical sense, I won't accept it.” Loadings for the final scale derived through Principal Components Analysis (PCA) ranged from 0.704 to 0.828, with its eigenvalue explaining 57% of the variance of the items. Internal consistency was demonstrated with Cronbach's Alpha (a)=0.878.

Days and weeks preceding the experience:

These items examined the events and emotions in participants' lives in the days and weeks before the psilocybin experience to identify the extent to which they were experiencing identity distress.

Two pages of data were collected. The first page activated participants' memory of this period through the prompt “Describe what was happening in your life in the days and weeks immediately preceding your experience.” The second page included 28 Likert-scaled items measuring state of psychological crisis measuring three a priori dimensions, of which only one was used within the model due to collinearity. That variable was:

Identity Distress

This measure which included four items to capture the state of turmoil or uncertainty representing readiness for change with items like “I′d felt wracked by doubt and uncertainty” and “I′d felt a desperate need to discover a new self.” The final internally consistent measure (α=0.891) explained 75.4% of the variance of the items with loadings from 0.841 to 0.883. Scale items were intermixed and also included three attention items to identify and exclude data from inattentive participants.

Onset of the Experience

The onset of the experience was measured in two pages. The first activated individuals' memory in open-ended questions about their expectations, preparation for their experience, and additional closed-ended questions about companions and location of their experience. On the second page, individuals were presented with 26 statements and four validation items related to their mental state at the time of the onset of their experience. All items were intermixed in five-item blocks and rated on a four-point scale for reasons stated above. The final scales included the following:

State of Surrender Scale

The final scale included 12 statements related to state of surrender (e.g., “I had stopped resisting and was ready to give up control”). A PCA found loadings on the State of Surrender scale ranging from 0.688 to 0.775, with an Eigenvalue of 6.48 and α=0.920.

Preoccupation Scale

The final scale included four items related to preoccupation with one's immediate life (e.g., “I felt a little bit rushed for time” or “I had a lot of things on my mind at the time”). A PCA produced loadings ranging from 0.674 to 0.789, and internal consistency of α=0.740.

During the Experience

Participants' experiences were measured in two pages on the survey. The first activated their recollection of the experience through an open-ended question asking them to describe their experience and specific fixed response questions about their experience, including whether their eyes were primarily open or closed and the extent to which they were talking or exposed to media with words during their experience. On the second page, subjects responded to 78 intermixed items on a 4-point Likert scale from the Mystical Experience Questionnaire (MEQ30) and 36 items from the Sacred Emotions Scale (SES), measuring the extent to which they felt “exuberance” and “dread” during the experience.

MEQ30

The MEQ30 is a validated scale consisting of 30 items systematically validated and utilized in psychedelic studies. Some of the items from the MEQ30 were adapted for this study so that they were semantically consistent with the other scale (e.g., changed “I felt certain that I′d encountered ultimate reality” to “Certainty that I′d encountered ultimate reality”), and used a 4-point rather than a 7-point scale for reasons described above. The scale measures four dimensions of a mystical experience: (a) Mystical; 15 items with α=0.957, (b) Ineffability; 3 items with α=0.775, (c) Timelessness/Spacelessness; 6 items with α=0.844, and (d) Positive Affect; 6 items with α=0.851. The total scale produced Cronbach α=0.967. All of the reliability scores from this sample were similar to internal reliability scores produced in the norming sample (MacLean), which ranged from 0.831 to 0.936 for the four factors and 0.957 for the total scale. The high reliability of the scales and consistency with the reliabilities in the norming sample suggest that the experience was measured effectively and consistently with the norming sample.
Sacred Emotions Scale (SES)

The SES developed by Burdzy (Sacred Emotions Scale (Thesis; 2014), Bowling Green State University, Kentucky) in collaboration with advisor Kenneth Pargament, consists of 41 items to measure the “emotional impact of an individual's experience with the sacred” (p. iii). Of the 41 items, only 36 were included in this survey study because a section containing one set of five items was unintentionally deleted from the online survey. Although the SES included items related both to exuberance and dread, the exuberance scale was strongly correlated with the MEQ30 (Pearson r=0.844); thus, it was not used in any of the analyses. Items used in this study were specifically related to the experience of dread, which consists of 12 items with a reliability of 0.883 in our sample compared with α=0.91 in the original norming sample (Burdzy). Items related to feelings of powerlessness (e.g., I felt completely insignificant,” “I felt small”), feelings of worry (e.g., “I felt afraid,” “I felt ashamed”), and behavioral representations of these feelings (e.g., [I felt like] turning away”). The exuberance scale, not used in this study, produced reliabilities of α=0.932; this compares with reliabilities of 0.93 in the original scaling by Burdzy and is suggestive of comparability of the scales across the samples.

After the Experience

Participants were asked to describe their current state in two pages. On the first page, they were asked whether they would take psilocybin again if they had a chance, and provided with an open text box in which to reflect on their experience. The second page offered 46 statements including 15 intermixed items adapted from the work of Griffiths et al., (Psychopharmacology 2006, 187:268-283) measuring

Positive Emotions, Negative Emotions and Spirituality to which subjects responded on 4-point Likert scales ranging from “Strongly Decreased” to “Strongly Increased.”

### Procedures

Data Collection

Data were collected on Amazon Mechanical Turk (MTurk), an online source in which individuals participate in survey studies for small cash compensation. Subjects were recruited through MTurk postings seeking 150 subjects who had had a recent experience with psilocybin (“magic mushrooms”) to participate in study titled “Psilocybin Study” Individuals were informed that they would be asked to participate in a lengthy survey taking approximately 40 minutes in which they would answer more than 250 questions about their experience. Individuals who were registered as MTurk Workers from the U.S. indicated their interest by accepting the job or the “hit.” If individuals accepted the “hit,” they were directed to a screening page. If they passed those screening items, described below, they were given a link and password to the informed consent followed by the actual survey.
Screening

Screening consisted of three close-ended questions to which affirmative responses brought them to the next screening page and eventually to the informed consent, and negative responses brought them to a page that stated “Sorry. Your response indicates that you are ineligible to participate in this study. Thank you for your time and interest.” After passing all of the screening items and agreeing to the informed consent, individuals were provided with a link and password for the actual survey. The number of individuals entering the actual survey exceeds the number requested because the “hit” remains open to more participants while current individuals are completing the survey; thus, an individual may be in the process of completing the survey when the “hit” closes.

A total of 323 individuals entered the screening page and were asked four questions to determine their eligibility. First, they were asked “Are you 18 years of age or older?” One subject was eliminated through this question. The next question was “Have you had a recent psilocybin experience?” An additional 17 participants were eliminated at this stage for saying no, and four non-respondents also left the survey at this point. Question 3 was “In what year was your psilocybin experience?” An additional 19 subjects who indicated that their psilocybin experience occurred before 2015 were eliminated at this stage. Finally, subjects were told “This is a very long survey, taking 35-45 minutes to complete, and we can offer only minimal compensation for your valuable time. Do you have 35-45 minutes to generously donate to this survey?” Eight subjects indicated that they did not have 35- 45 minutes to donate and were taken to an exit page. An additional 15 subjects answered all of the screening questions but failed to complete the Informed Consent, yielding a total of 259 individuals who completed the informed consent and were taken to the screen where the survey password was provided. Of these, 185 individuals used the password to enter the survey.

The final screening process eliminated participants who did not accurately respond to at least 10 of the 12 attention items; this step eliminated 23% (n=42) of those who had entered the survey. The mean number of correct attention items in the final sample was 11.5 of 12 (SD=1.02), and the mean for the non-selected group was 4.69 (SD=3.36) attention items answered correctly. The final sample consisted of 143 valid responses with 81 males and 62 females (43%), and an average age of 31.1 (SD=9.64), with ages ranging from 18 to 62 years. Approximately 14% of participants had a graduate degree or some graduate education, 29% had completed a Bachelor Degree, 40% reported that they had some college, 11% had completed an Associate Degree, and 11% had not yet continued their education beyond a high school diploma. Approximately 50% of respondents reported that formal religion had been important or very important in their childhood families, but only 23% reported that it was still important in their lives today; in fact, 61% reported that formal religion was not at all important in their lives today. In contrast, 51% reported that spirituality was important or very important in their lives today, and only 29% reported that spirituality was not at all important. The majority of participants (78%, n=112) had previous experience with psychedelic drugs. Of those who had used psychedelic drugs previously, 25% had used 1-2 times, 38% had used 3-5 times, 22% had used 6-10 times, and 15% had used more than 10 times in the past ten years.
Statistical Method

Hierarchical linear multiple regression with data entered in blocks was used to (1) determine the predictive power of hypothesized variables on dependent measures of ME and Dread, and (2) to identify a potentially mediating role of ME on long-term positive change. In all cases, blocks were entered into the hierarchical regression model in logical order of their appearance in and influence on an individual's life. Block 1 included demographic factors (Age, Sex, and Education). Block 2 included trait factors (TAS, mental barriers, REI). Block 3 included prior experience with psychedelics (Pex) and identity distress. Block 4 included two measures of mental state at the onset of the experience (surrender and preoccupation), and Block 5 included relevant setting factors (group size and proportion of time with eyes open). Predictors were narrowed through two processes: (1) eliminating blocks that did not present any significant changes to R2, and (2) eliminating predictors within blocks if collinearity was present. Each model was then examined for collinearity problems or other violations of the assumptions for regression. In all cases, all of the observations were independent and met the criteria of 2.0 from a Durbin-Watson statistic of 1.910. Examination of the plot of studentized residuals versus unstandardized predicted values revealed homoscedasticity of variance. Multicollinearity is not present in the model, as demonstrated through the fact that correlations among all the predictors used in the final model have r values less than 0.7, and Tolerance values are well over 0.1, ranging from 0.564 to 0.769. One potential outlier was detected in which the predicted value was more than 3 SD from the expected value; the outlier was left in the model because no rationale could be offered for deleting it. No cases had leverage values greater than 0.2, nor did their Cook's Distance value exceed 1. Finally, residuals appeared to be normally distributed, as evidenced through (a) examination of the histogram associated with the regression standardized residual, which produced a mean near to 0 and a SD near to 1, and (b) examination of the P-P plot.

### Results

Correlations among new and previously validated scales

Because new constructs were developed, a preliminary look at their construct validity is provided by examining a correlation matrix of all the variables measured. Although the items were validated within a broader sample, the correlation matrix includes only the psilocybin sample used in this study. The matrix appears below as Table 1. Several interesting relationships may be noted. First, age correlated inversely with both dread and ME030 scores, but the correlation was substantially stronger for dread at −0.213. Second, a relationship exists between state of surrender and REI. Third, identity distress is associated inversely with eyes open, suggesting that individuals with high identity distress were more likely to have eyes closed for the majority of the experience. Identity distress is also associated positively with both preoccupation and state of surrender, and with both dread and ME030. Finally, it may be noted that males were more likely to have previous experience with psychedelics (Pex) and greater mental barriers.

TABLE 1
Correlations among predictor and dependent measures.

| | Age | Edu | Sex | Pex | TAS | MB | REI | ID | Eyes | Grp | Pre | Sur | Dread | MEQ30 |
|---|---|---|---|---|---|---|---|---|---|---|---|---|---|---|
| Age | 1 | | | | | | | | | | | | | |
| Edu | .098 | 1 | | | | | | | | | | | | |
| Sex | −.122 | −.062 | 1 | | | | | | | | | | | |
| Pex | .028 | −.060 | .208* | 1 | | | | | | | | | | |
| TAS | −.077 | .018 | −.080 | .243** | 1 | | | | | | | | | |
| MB | −.241** | −.020 | .251** | −.151 | −.114 | 1 | | | | | | | | |
| REI | −.056 | −.160 | −.136 | .079 | .527** | −.172* | 1 | | | | | | | |
| ID | −.148 | .047 | .021 | .157 | .341** | .101 | .089 | 1 | | | | | | |
| Eyes | .064 | .031 | .066 | .041 | −.033 | −.153 | −.063 | −.232** | 1 | | | | | |
| Grp | −.170* | .073 | −.202* | .02 | −.006 | −.034 | −.012 | −.091 | .097 | 1 | | | | |
| Pre | −.030 | .148 | −.085 | .034 | .230** | .143 | −.051 | .492** | −.16 | −.048 | 1 | | | |
| Sur | .116 | −.030 | −.056 | .278** | .480** | .040 | .268** | .370** | −.046 | −.070 | .126 | 1 | | |
| Dread | −.213* | .087 | .015 | −.056 | .322** | .153 | −.003 | .523** | −.197* | .021 | .648** | .020 | 1 | |
| MEQ30 | −.033 | −.030 | .000 | .262** | .605** | −.144 | .372** | .372** | .022 | −.087 | .063 | .727** | .070 | 1 |

*p < ;05;
**p < .01.
Note:
Sex was coded Female = 1; Male = 2; thus, higher numbers correspond with male; “Pex” = Previous experience with psychedelics; “TAS” = Tellegen Absorption Scale; “MB” = Mental barriers, “REI” = Rational Emotive Inventory; “ID” = Identity Distress in days and weeks before the experience; “Eyes” = the proportion of time eyes were open during the experience; “Grp” = Group size during the psychedelic experience; “Pre” = Preoccupation with time or tasks at the onset of the experience; “Sur” = Surrender at the onset of the experience.

Predicting ME

Examination of the statistics for the initial model above showed that the items in Block 1 and Block 5 did not produce correlation coefficients of .3 or greater for any of the relationships, explaining 2% of the variance (Block 1) and producing an R2 change of 0.009 for Block 5. All of the items in Blocks 1 and 5 were eliminated from the model. All of the remaining Blocks added significantly to the model; however, neither REI nor Pex added substantially to the model with standardized beta weights <0.1. Therefore, both of these variables were also eliminated.

The final regression model included Block 1: absorption; Block 2: mental barriers; Block 3: identity distress; Block 4: surrender and preoccupation at the onset of the experience. As is shown in Table 2, the overall regression model explained 63.6% of the variance in ME. Model 1 shows that absorption alone explained 36.1% of the variance in ME. The explanatory power increased slightly but significantly to 39.5% with the addition of mental barriers and identity distress in Models 2 and 3. With the addition of preoccupation and surrender in Model 4, the explanatory power increased markedly to explain a total of 63.6% (adjusted R2) of the variance in ME. In this final model, all of the predictors were significant, with the strongest Beta weights attributed to surrender (β=0.549) and to absorption (β=0.309). The direction of all of the relationships were as expected.

TABLE 2
Linear Regression Predicting ME with Trait and State Factors

| | | B | SE | β | t | Sig. | Adj. R² |
|---|---|---|---|---|---|---|---|
| Model 1 | (Constant) | .983 | .225 | | 4.368 | .000 | .361 |
| | Absorption | .698 | .077 | .605 | 9.022 | .000 | |
| Model 2 | (Constant) | 1.179 | .284 | | 4.158 | .000 | .363 |
| | Absorption | .688 | .078 | .596 | 8.843 | .000 | |
| | Mental barriers | −.071 | .063 | −.077 | −1.135 | .258 | |
| Model 3 | (Constant) | 1.168 | .276 | | 4.227 | .000 | .395 |
| | Absorption | .604 | .081 | .523 | 7.443 | .000 | |
| | Mental barriers | −.098 | .062 | −.106 | −1.588 | .115 | |
| | Identity Distress | .136 | .047 | .204 | 2.905 | .004 | |
| Model 3 | (Constant) | .745 | .228 | | 3.272 | .001 | .636 |
| | Absorption | .357 | .070 | .309 | 5.120 | .000 | |
| | Mental barriers | −.118 | .049 | −.127 | −2.435 | .016 | |
| | Identity Distress | .093 | .042 | .139 | 2.211 | .029 | |
| | Preoccupation | −.114 | .053 | −.128 | −2.158 | .033 | |
| | Surrender | .537 | .059 | .549 | 9.114 | .000 | |

Dependent Variable: Mystical Experience (ME)

Dread

Table 3 displays data for a regression model predicting dread. The initial model was developed with all of the predictor variables used to predict ME and, like the models predicting ME, Blocks 1 and 5 offered no significant increase to the explanatory power of the model; thus, both blocks were eliminated. As is shown in Table 3, the overall regression model explained 53.3% of the variance in dread. The trait variable absorption explained 9.8% alone, as shown in Block 1, and mental barriers explained an additional 3% of variance when added in Block 2. The addition of identity distress in Block 3 added substantially to the explanatory power (R2 change=17.3%), and the addition of preoccupation and surrender variables in Block 4 added even more to prediction, explaining a total of 53.3% (adjusted R2) of the variance in dread. In the final model, all of the predictors except mental barriers were significant in expected directions. The strongest Beta weights were for preoccupation (β=0.462), while identity distress, surrender, and absorption predicted similarly with Beta weights of 0.301, −0.277, and 0.258, respectively.

TABLE 3
Linear Regression Predicting Dread with Trait and State Factors

| | | B | SE | β | t | Sig. | Adj. R² |
|---|---|---|---|---|---|---|---|
| Model 1 | (Constant) | .927 | .272 | | 3.409 | .001 | .098 |
| | Absorption | .378 | .093 | .322 | 4.045 | .000 | |
| Model 2 | (Constant) | .426 | .337 | | 1.265 | .208 | .128 |
| | Absorption | .404 | .092 | .344 | 4.365 | .000 | |
| | Mental barriers | .182 | .075 | .192 | 2.436 | .016 | |
| Model 3 | (Constant) | .401 | .302 | | 1.328 | .186 | .299 |
| | Absorption | .216 | .089 | .185 | 2.437 | .016 | |
| | Mental barriers | .122 | .068 | .129 | 1.796 | .075 | |
| | Identity Distress | .303 | .051 | .448 | 5.918 | .000 | |
| Model 3 | (Constant) | .315 | .262 | | 1.202 | .231 | .533 |
| | Absorption | .302 | .080 | .258 | 3.767 | .000 | |
| | Mental barriers | .092 | .056 | .097 | 1.638 | .104 | |
| | Identity Distress | .204 | .048 | .301 | 4.230 | .000 | |
| | Preoccupation | .418 | .061 | .462 | 6.883 | .000 | |
| | Surrender | −.275 | .068 | −.277 | −4.065 | .000 | |

Dependent Variable: Dread

Positive Response

The regression model to predict positive response was created using the same predictors as for ME, but adding ME itself in a separate block to explore its impact as on positive change. ME was entered as a fifth block predictor within the regression model in order to test the hypothesis that ME mediates the relationship between other predictors and positive change. The mean positive change score was 3.58 (SD=0.65), with higher scores representing increases in positive emotions and decreases in negative emotions. Tolerance statistics suggest a potential problem with collinearity, with variance inflation (VIF) factors for ME (VIF=2.774) slightly exceeding optimal levels. However, because a mystical state can mediate the relationship between the predictors and positive change, and the collinearity level was known from the ME regression reported above, the model was retained.

As is shown in Table 4, the variables that were originally used to predict ME explained only 22% of the variance in positive change, but when ME was added to the model as a predictor, the explanatory power increased significantly to 37.8%. Before adding ME, mental barriers (β=0.165), and surrender (β=0.318) were both significant predictors in a model explaining 22% of the variance in positive change. When ME was added to the model, it explained 37.8% of the variance in positive change, with ME having a large Beta weight (β=0.661), and eliminating the significance of every other predictor in the model.

TABLE 4
Linear Regression Model for Positive Change

| | Model 1 (without ME) | | Model 2 (with ME) | |
|---|---|---|---|---|
| | β (t-value) | | β (t-value) | |
| (Constant) | −.086 | (−.998) | −.002 | (−.025) |
| Preoccupation | | | | |
| Mental barriers | −.165 | (−2.16)* | −.081 | (1.16) |
| Identity Distress | −.022 | (−.236) | −.114 | (−1.36) |
| Absorption | .233 | (2.64) | −.029 | (−0.33) |
| Surrender | .318 | (3.61)** | −.045 | (0.41) |

| | | | |
|---|---|---|---|
| Mystical Experience | — | .661 | (5.91) *** |

| | | |
|---|---|---|
| Adi. R² | .220 | .375 |

*** p < 0.001;
**p < 0.01;
*p < 0.05

Consistent with the prediction that ME would mediate the relationship of other variables with positive change, all other direct paths to positive change were muted (became non-significant) when ME was entered into the model. Drawing from the logic of path modeling and mediation (e.g., Maruyama, Basics of Structural Equation Modeling, 1998, Sage Publications, Thousand Oaks, Calif.), if ME were a mediator, it would be positioned between other predictors and positive change. In such a model, for each of the other predictors, their mediated (indirect) effect is the product of the path to ME and the path from ME to positive change. For example, the indirect path for surrender is 0.530*0.653=0.350, a substantial mediated effect. Subtracting that mediated relationship from its direct path when ME is not included (0.301) yields its path in the final regression of −0.049. Using similar logic, the indirect paths for the remaining four variables are: absorption (0.164), mental barriers (−0.102), identity distress (0.094), and preoccupation (0.090). Not all mediated effects were large, but they were each large enough that no direct path remained significant.

Predicting Complete Mystical Experience

Although this model predicted strength of ME as measured on a continuum, ME is often measured as complete or less than complete using threshold scores. Barrett et al. (Journal of Psychopharmacology 2015, 29:1182-1190), for example, used 60% on all of the subscales as the threshold for a complete ME in their work. Because the 4-point scale used in our study allowed for far less variability in scores when compared with the 10-point scale in the MEQ30, ME was dichotomized at the 50% point such that those individuals reaching >2.5 (of the maximum possible mean of 4) on all four subscales were identified as having had a complete ME. Within this sample, 58% met criteria for having a complete ME. Proportions experiencing complete ME did not vary by sex, but a chi-square revealed that a significantly larger proportion of experienced psychedelic users had a full ME (63%, n=70) when compared to novice users (42%, n=13) (x2=4.216; p=0.04).

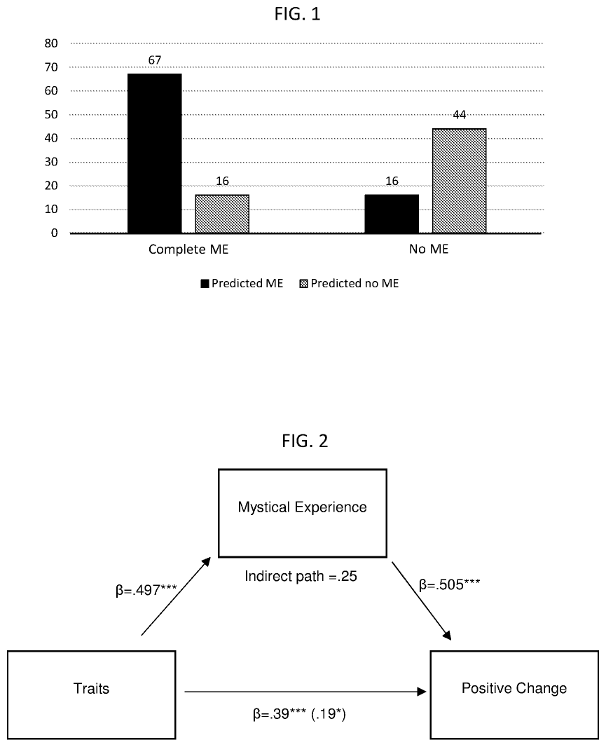

Because 58% of individuals surpassed the threshold for complete ME, the unstandardized predictors for this model were saved for each individual and at dichotomized at the 42nd percentile (100% minus 58%) to be proportionally aligned with the frequencies for complete ME. It is shown in FIG. 1 that the model correctly predicted complete mystical experience 80.7% of the time and absence of ME 73.3% of the time. In other words, the model predicted that 83 individuals would have ME, and 67 of those actually had ME. Conversely, it predicted no or incomplete ME for 60 participants, and 44 of these in fact had no or incomplete ME.

Finally, Complete ME significantly predicted significantly higher levels of positive change after the psychedelic experience. Table 5 shows that those experiencing a complete ME experienced significantly larger positive change than those with no/incomplete ME with an effect size (Cohen's d) of 0.84.

TABLE 5
T-test of Positive Change for Complete vs. No/Incomplete ME

| | N | Mean | SD | SEM | t value |
|---|---|---|---|---|---|
| Complete ME | 83 | 3.8348 | .60308 | .06620 | 6.378*** |
| No/Incomplete ME | 60 | 3.2274 | .53044 | .06848 | |

### Discussion

Trait factors significantly predicted ME in expected ways. When entered in Block 1 (Table 2), absorption alone explained more than one-third of the variance in ME, producing explanatory power similar to that found in the meta-analysis by Studerus et al. (PLoS ONE 2012, 7), in which explanatory power of absorption on dimensions of ME ranged from 20-40%. This suggests that the population in our study resembled the population in other controlled studies, despite the differences in the conditions of the psychedelic administration. When mental barriers was added to the model in Block 2, the explanatory power increased slightly, with mental barriers inversely related to MEs as predicted by Maslow (Journal of Transpersonal Psychology 1970, 2(2):83-90).

State factors of surrender at the onset of the experience and identity distress in the days and weeks before the experience would override trait factors in predicting ME. This model suggests an especially strong predictive power for surrender. While identity distress added significantly but slightly to the exploratory power of the model, the addition of surrender in the final model nearly doubled the explanatory power of the earlier predictors to explain almost two-thirds of the variance in ME. Thus, an ability to set aside one's physical interests and goals, place oneself in a fully receptive state, and commit or surrender to the psychedelic session fully and completely for whatever may be learned or received from it increases the extent to which MEs can be experienced. Further, a state of identity distress in the days and weeks preceding the experience in conjunction with this state of surrender at the onset strengthened the likelihood of ME during the psychedelic session. This relationship between identity distress and surrender to produce ME parallels the mental states in which psychedelic substances are used in traditional or ritual practices.

The models also suggests that ME mediates the psychedelic experience and positive response (e.g., transformative positive outcomes). While the variables described earlier predicted one-fifth of the variance in positive change independently of ME with beta weights in expected directions, the addition of

ME to the model increased the explanatory power to 37.5% and muted all of the other predictors. Thus, we can conclude that absorption, identity distress, and a state of surrender in isolation or in combination do little to produce transformative impact; it is the ME in conjunction with the preparatory states and not the preparatory states themselves that produce the transformations in mood, outlook, and spirituality—and of course, in the present context, psilocybin itself is the key variable producing MEs.

Finally, these results suggested that a similar model would inversely predict an adverse psychedelic experience. A blocked, anxious, or “dread-ridden” psilocybin experience was significantly more likely when its onset was marked by worry and uncertainty about one's own agenda, or a state of preoccupation. This dread experience was still more likely when the preoccupied state was maintained in conjunction with reduced ability to surrender to the experience, a preexisting state of identity distress, higher absorption scores, and stronger mental barriers. The state of preoccupation with one's own agenda or time at the onset of the experience served as the strongest predictor. Thus, one might also suggest that preoccupation represents a cognitive attachment to the details of one's life events, whether good or bad, that opposes the necessary receptive state of surrender that is optimal for ME. Such preoccupation could also be linked to a desire to maintain “ego-integrity” and a resistance to allowing the ego to disintegrate or dissolve. Clearly, one who remains busily mentally engaged with the details and tasks of one's life is in a state of egotism that is inconsistent with a willingness to abandon those details in favor of an unknown new terrain. The accompanying relationship between mental barriers and dread, while nonsignificant in the final model, may represent an attachment to one's ideas, beliefs, or preconceived notions (that characterize one's ego) and that work against the occurrence of MEs, promoting instead aversive experiences.

The trait of absorption predicted ME during the psychedelic experience, producing standardized coefficients in the regression model before and after state of surrender and preoccupation were added to the model (β=0.523 and 0.309, respectively). Absorption also predicted dread in this model with unstandardized coefficients of 0.185 and 0.258 before and after preoccupation and surrender were added to the model. High absorption may be related to (e.g., correlated with) high-suggestibility, as described by Carhart-Harris et al. (Psychopharmacology 2015, 232:785-794), and that this latter trait may contribute additional explanatory value if included in subsequent studies of psychedelic predictors.

Absorption also correlated positively with both the state of surrender that predicted ME and with the state of preoccupation that preceded Dread during a psilocybin session. The state of surrender itself is a relatively passive experiential state; as such, high-absorption individuals may more easily generate and ease into this state that appears to enhance ME. Conversely, if the state of preoccupation is laden with the instrumental goals worries, as suggested by the items in the scale, these concerns may in turn be amplified for high-absorption individuals and increase the likelihood of a dread experience. This matter may have implications for controlled studies of psychedelics, where participants are sometimes asked to complete cognitively demanding tasks.

This model thus supports the general framework for the importance of the conditions of mental set under which the psychedelic is taken such as suggested in indigenous ritual and religious conversion history. A state of complete commitment or surrender to the experience increased the likelihood of ME, particularly when accompanied by a preceding state of uncertainty. Conversely, mental barriers causing one to reject illogical or non-rational experiences reduced the likelihood of ME. A different mental state, in which one is busily engaged with the details of one's life, feeling deeply uncertain but unable to surrender into the psychedelic experience, predicted an adverse response. The trait of absorption served as an amplifier for both positive and negative psychedelic experiences. The results suggest that efforts to promote states of psychological readiness and surrender in advance of psychedelic ingestion, are important and well-advised.

### Example 2. Validation of Predictors Identified in Example 1 and Identification of New Predictors

The purpose of this study was to replicate the findings of Example 1 in a separate sample from which more information could be collected related to dosage and setting factors. Such a replication, using different measures of adverse and mystical experience, sought to lessen the limitations of retrospective data and strengthen the coherence of the constructs surrender and preoccupation as predictors of response to psilocybin. The study of this Example thus serves three key purposes: (1) to test a conceptual replication of the regression model predicting response to psilocybin found in Example 1 on a distinct second sample using different dependent measures, (2) to test the importance of additional hypothesized predictors on mystical and adverse experience, and (3) to explore the relationship between optimal response to psilocybin and long-term positive change.

### Methods

Participants were U.S. residents recruited through Amazon Mechanical Turk (MTurk; an online source in which individuals participate in survey studies for small cash compensation) who had ingested psilocybin within the past twelve months. The final screened sample consisted of 183 valid responses with 97 males (53.0%) and 85 females (46.4%) (1 missing), and an average age of 31.9 (SD=9.43) ranging from 18 to 70 years. Seventeen percent (n=31) of participants had a graduate degree or some graduate education, 36% (n=11) had completed a bachelor's degree, 21% (n=39) reported that they had some college, 13% (n=22) had completed an associate degree, and 14% (n=25) had not yet continued their education beyond a high school diploma.

Twenty-seven percent of participants (n=49) reported no previous experience with psilocybin, and 35 (71%) of those who were naïve to psilocybin also had no experience with any other psychedelic drug. The age of experienced participants' first psychedelic session ranged from 12 to 53 years, with the majority (51.4%) having their first experience before the age of 21.

The psilocybin sessions on which this study was based occurred within 12 months of data collection, including 24 participants (13.2%) whose session was 10-12 months prior, 37 (20.3%) 7-9 months prior, 65 (35.5%) 4-6 months prior, and 57 (31.2%) 1-3 months prior.
Survey Instrument

The online survey consisted of 286 items in six sections on an online survey tool Survey

Monkey. The six sections included background items, trait measures, prior state (i.e., life situation before psilocybin session), proximal state (i.e., emotions and motivation at ingestion), intrasession measures (i.e., the experience itself), and current state. Scaled items in all but the long-term outcomes used four-point Likert scales ranging from 1=Strongly Disagree to 6=Strongly Agree. Open-ended questions designed to activate participants' relevant recollections were completed before responding to scale items related to prior state, proximal state, intrasession, and current status. Measures utilized in the regression models include the following;

Tellegen Absorption Scale (TAS).

A trait or disposition “for having episodes of “total” attention that fully engage one's representational (i.e., perceptual, enactive, imaginative, and ideational) resources.” (Tellegen and Atkinson, Journal of Abnormal Psychology 1974 83(3), 268-277). 18 of the 34 original items were included in this study, with Cronbach's α=0.88 in the norming sample (Green and Lynn, Journal of Clinical and Experimental Hypnosis 2010 59:103-121) and α=0.91 in this sample.

Barriers

A new 5-item trait measure of the extent to which individuals tend to reject information or experiences that are not logical, rational, or scientific with Cronbach's α=0.88 in this sample, similar to α=0.87 in Example 1.

Deservingness

A new 5-item trait measure or the extent to which individuals believe they deserve the best and richest because they earned them (e.g., “I have a right to use as much water or fuel as I want.”) with Cronbach's α=0.72.

Confusion

A new 5-item measure of a state of uncertainty about who one was or where one was going, included among 30 additional items measuring crisis and thriving in the days and weeks before ingestion (“Prior State”). Cronbach's α=0.91, similar to α=0.92 in Example 1.

Motivations

A new measure of participants' motivations for taking psilocybin. Items were generated from analysis of open-ended responses in Example 1 and statistically reduced to a 14-item four-factor model including spiritual motivations, norm motivations, pleasure motivations, and betterment motivations, as shown in Table 6, below.

TABLE 6
Pattern matrix from principal axis factoring showing loadings of variables
associated with reasons for having a psilocybin experience.

| Items | 1-Spiritual | 2-Norms | 3-Pleasure | 4-Betterment |
|---|---|---|---|---|
| I wanted to be better able to give to others. | .892 | .103 | −.037 | −.109 |
| I wanted greater capacity to love all beings. | .830 | −.015 | .035 | .069 |
| I wanted to help make the world a more peaceful | .775 | .043 | .064 | −.030 |
| place. | | | | |
| I wanted to find my soul or the divine presence. | .658 | −.122 | −.050 | .187 |
| I wanted to feel one with the universe. | .598 | −.066 | .210 | .138 |
| Everybody else was taking it, so I did too. | −.056 | .881 | −.070 | .062 |
| I wanted to join my friends who were doing it. | −.069 | .749 | .121 | −.116 |
| I wanted to show others that I could handle it. | .165 | .642 | −.012 | .094 |
| I wanted to experience all kinds of sensations. | −.024 | −.053 | .836 | −.013 |
| I wanted to see amazing visions and colors. | .011 | .139 | .717 | −.003 |
| I wanted to have a really cool or beautiful | .144 | −.056 | .662 | .066 |
| experience. | | | | |
| I wanted to have less anxiety or fear. | −.019 | .089 | −.064 | .897 |
| I wanted to change or improve my outlook on life. | .211 | −.057 | .005 | .729 |
| I wanted to be happier and more peaceful. | −.020 | −.055 | .343 | .599 |

Extraction Method: Principal Axis Factoring. Rotation Method: Oblimin with Kaiser Normalization. Rotation converged in 5 iterations. Four factors explained a total of 64.8% of variance in the items. Bold font highlights items loading on each factor.

Words

The extent to which spoken words in the form of conversation or background language were present during their psilocybin session. This was measured by summing responses to two 0-100% slider scales for “language” (percent of time spoken language including song lyrics was present in background or foreground) and “conversation (percent of time they were actively conversing).

Apprehension

A single item asking individuals how apprehensive they felt immediately prior to ingesting psilocybin on a 4-point scale from “not at all apprehensive,” to “Very apprehensive.”

State of Surrender (SoS) and State of Preoccupation (SoP)

New measures of mental state at the time of psilocybin ingestion. Surrender (10 items, Cronbach's α=0.92) is a readiness to accept whatever happens, whether good or bad, without resisting or fighting or struggling, and preoccupation (4 items, α=0.757) is a measure of mental busyness with one's life events and tasks.

Mystical-OBN and Adverse-DED

Measures of intrasession experience based on items from six subscales of the 5D-ASC (Dittrich et al., A Pharmacopsychiatry 1998, 31:80; Studerus et al., PLoS ONE 2010, 5). Mystical-OBN used a mean of items measuring Oceanic Boundlessness (Studerus et al., PLoS ONE 2012, 7): “experience of unity”, “spiritual experience”, “blissful state”, and “insightfulness”. Adverse-DED was a mean of items associated with Dread of Ego Dissolution (Studerus et al., PLoS ONE 2012, 7): “impaired control and cognition”, and “anxiety.” Scores for Mystical-OBN ranged from 1-5.92 (M=3.87, SD=1.16; α=0.93), and scores for Adverse-DED ranged from 1-5.47 (M=2.25, SD=0.98, α=0.91).

Ego Dissolution Inventory (EDI)

The EDI measures ego dissolution, the “reduction in the self-referential awareness that defines normal waking consciousness” (Nour, et al., Frontiers in Human Neuroscience 2016, 10:269) in two factors: ego dissolution and ego inflation. Only ego dissolution was included in this study, with Cronbach's α=0.87 in our sample compared to α=0.93 in the norming sample.

Positive Change and Negative Change

Scale items measuring change based on pairs of adjectives adapted from Griffiths (2006). Individuals used a 5-point scale to indicate whether they had experienced a decrease, no change, or an increase in the characteristics. Positive change was calculated with the mean of nine items related to interpersonal traits, intrapersonal characteristics, and attitudes (Cronbach's α=0.929). Negative change was the mean of three items (α=0.883).

### Procedures

Data Collection

Data were gathered from participants registered in Amazon Mechanical Turk (MTurk). The recruitment post sought 230 U.S. residents who recently had taken psilocybin and could respond to a lengthy survey “Psilocybin Experience” for compensation of $1.50. A total of 624 individuals entered the screening page, and 318 passed screening, completed the informed consent, and were given the link and password to the survey. Of those entering the survey, 135 were eliminated because they did not complete the entire survey or because they did not respond correctly to at least 12 of the 14 validation items.
Statistical Approach

Replicating earlier model

Linear backwards regression with predictors identified from Example 1 was used to determine whether the model fit reasonably well in a second sample with alternate measures of mystical and adverse experience, the 5D-ASC.

Extending the model

An updated model with additional trait, expectation, and setting predictors was generated in two steps: (1) All potential predictors were entered into a hierarchical regression model predicting mystical-OBN and adverse-DED in blocks: Block 1: (age, education, sex), Block 2: Traits (absorption, barriers, entitlement, openness, rigidity, deservingness); Block 3: Motivations (spiritual, betterment, norms, pleasure); Block 4: Setting (preferred amount, people, language, eyes closed/open, conversation); Block 5: Prior State (confusing, thriving); Block 6: Proximal state (surrender, preoccupation, apprehension). (2) Any variable producing a Beta weight with p<0.1 in any block was included in a backwards regression to identify the best predictors in a final holistic model.

Mystical-OBN had slight negative skew but was retained without transformation. Adverse-DED had positive skew and was log-transformed to reduce skew, after which it displayed an acceptable distribution. Three outliers in the mystical-OBN distribution were examined and retained because they could not be eliminated purposefully. All of the other variables had acceptable statistical properties. Missing data were not present because responses were required for each item.

Examining plausible mediation relationships

Analyses of the impact of mystical experience on long-term outcomes were conducted in two ways: (1) independent t-test comparisons of each long-term outcome based on a complete or not complete mystical experience, and (2) a path analysis of the mediating role of mystical experience on positive change. A “complete” mystical experience was calculated in alignment with the description in Studerus (PLoS ONE 2012, 7), in which those individuals experiencing more than 70% of the possible points on the OBN scale were rated as having had a complete experience.

A path analysis examined the plausibility of a mediating role of mystical-OBN on the relationship between traits and positive change in four steps: (1) Composite traits variables were entered for each outcome based on the traits that best predicted it; (2) Hypothesized predictors, mediators, and outcomes were entered into linear regression models to test the relationships stipulated by Kenny (Kenny, et al., Data analysis in social psychology vol 1; The Handbook of Social Psychology 1998, 4 edn.); (3) Data from plausible relationships were imported into LISREL for goodness of fit test using SEM statistics as recommended by Maruyama (Basics of Structural Equation Modeling, 1998, Sage Publications, Thousand Oaks, Calif.) and others; (4) A Sobel test was conducted to determine whether the relationships within the model supported a mediation model.

### Results

Replication of Predictive Model

Mystical Experience

In Sample 1 (S1; of Example 1), mystical experience was measured with the MEQ-30. In the current sample (S2), it was measured by the subscales of the 5D-ASC. The best predictors that explained 65.7% (Adj. R²=0.644) of the variance in mystical-MEQ for S1 explained 53% of the variance in mystical-OBN (Adj. R²=0.518) in S2. SoS was the strongest predictor for mystical experience in both samples (β=0.607 in S1 and 0.469 in S2), and absorption was second strongest in both samples (β=0.308 in S1 and 0.369 in S2). Words also predicted significantly and negatively in both samples (β=0.130 in

S1, −0.143 in S2). The other predictors significant in S1, barriers and age, did not produce significant Beta weights in S2. When compared by sex, the S2 model predicted 60.4% of the variance for males but only 43.8% of the variance for females, with words retained a significant inverse predictor for males only.

Adverse Experience

In S1, adverse experience was measured with the dread subscale of the Sacred Emotions Scale Burdzy, Sacred Emotions Scale (Thesis; 2014), Bowling Green State University, Kentucky, and referred to as adverse-SES. In S2 it was measured by two subscales of the 5D-ASC (Dittrich et al., A Pharmacopsychiatry 1998, 31:80; Studerus et al., PLoS ONE 2010, 5) and named adverse-DED. The same predictors that explained 56% (Adj. R²=0.545) of the variance in adverse-SES in S1 explained 32.5% of the variance in adverse-DED in S2 (Adj. R²=0.306). In both samples, SoP was the strongest predictor (β=0.440 in S1 and 0.424 in S2), and confusion was second (β=0.335 in S1 and 0.223 in S2). In S2, neither age, absorption, nor surrender had significant predictive power, whereas they all were significant in S1. When compared by sex, the model explained 28.6% of the variance for males and 39.0% for females, with age retained as a significant inverse predictor for females only. Females (M=30.9 years) in this sample were not significantly younger than males (M=32.7 years) (one-tailed t-test=1.293, p=0.89).

Revised Model Predicting Mystical Experience

Because additional hypothesized predictors were measured in S2, all possible predictors were entered into a linear regression in blocks to identify clusters that contributed to predictions of mystical-OBN. Demographic factors in Block 1 (age, education, sex) explained no variance in mystical-OBN (R²=0.010). Trait variables in Block 2 (openness, deservingness, barriers, rigidity, absorption, and entitlement) increased the R² significantly to 0.312 (Adj. R²=0.276), and the addition Prior State factors in Block 3 (confusion, thriving) increased the explanatory power to .333 (Adj. R²=0.290). Motivations for having the psychedelic experience in Block 4 (spiritual, norms, and pleasure) increased the R² to 0.493 (Adj. R²=0.439). The Setting factors in Block 5 (time with eyes closed, number of people present, words, and amount preferred) did not add significantly to the model, but the Proximal State or “Set” factors in Block 6 (surrender, apprehension, and preoccupation) significantly increased the R² to 0.63 (Adj. R²=0.58). Table 7 shows explanatory power for each block.

The best-fitting model included five predictors explaining 61% of the variance in mystical-OBN (Adj R²=0.599): State of surrender (⊖=0.453, p<0.001) was the strongest predictor, followed by spiritual motivations (β=0.289, p<0.001), absorption (β32 0.247, p<0.001), deservingness (β=−0.162, p<0.01), and apprehension (β=−0.117, p<0.05). See Table 7, below.

TABLE 7
Backwards stepwise regression predicting mystical-OBN

| | Unst. Coeff. | | Std. Coeff. | | | |
|---|---|---|---|---|---|---|
| Model | B | Std. Err. | Beta | T | Tolerance | VIF |
| (Constant) | .884 | .308 | | 2.871 | | |
| 2_Absorption | .296 | .067 | .247 | 4.402*** | .698 | 1.432 |
| 2_Deservingness | −.173 | .052 | −.162 | −3.366** | .946 | 1.058 |
| 3_Spiritual motivations | .219 | .044 | .289 | 4.999*** | .658 | 1.519 |
| 6_Apprehension | −.140 | .058 | −.117 | −2.418* | .944 | 1.059 |
| 6_Surrender | .453 | .054 | .453 | 8.412*** | .759 | 1.318 |

*p < .05,
**p < .01,
***p < .001.

A suppressor effect in which surrender (X2) appeared to serve as a suppressor for the trait deservingness (X1) in predicting mystical-OBN (Y) can be detected in this model. The zero-order correlation (r) between X1 and Y (r_{x1y}) was −0.036 (ns); rₓ₁ₓ₂=0.202 (p<0.05), and r_{x2y}=0.652 (p<0.001). Despite the negligible relationship between X1 and Y, X1 becomes a significant inverse predictor when X2 was entered into the model, as shown in. Further, in a model using only X1 and X2 to predict Y, it can be seen that (β_{X2}=0.688)>(r_{S2Y}=0.652), and (β_{X1}=−0.175)>(r_{X1Y}=−0.036), and that R² increases significantly from 0.426 to 0.455 (Adj. R²=0.423 to 0.449) with the addition of X1.

Comparisons by Sex

The models were similar by sex, but greater total variance in mystical-OBN was explained for males (R²=0.657; Adj. R²=0.642) than for females (R²=0.556; Adj. R²=0.527). Among the variables, state of surrender was the strongest predictor for both males and females (β=0.466 and 0.441, respectively), followed by spiritual motivations (β=0.321 and 0.310, respectively). The traits absorption (β=0.215 and 0.244) and deservingness (β=−0.233 and −0.110) also predicted for both sexes, but the set variable apprehension (β=−0.210) added significantly and inversely to prediction for females.

Comparisons by Spiritual Motivations

To explore the impact of spiritual motivations, the regression model was separately generated for individuals higher and lower in spiritual motivations after eliminating spiritual motivations as a predictor. More of the variance was explained for those lower in spiritual motivations (R²=0.519) than for those high in spiritual motivations (R²=0.406). For both high and low spiritual motivation groups, surrender was the strongest predictor (β=0.467 and 0.578). Absorption (β=0.236 and 0.364, p<0.001) and deservingness (β32 −0.231 and −0.173, p<0.05) were also retained as significant predictors; age did not predict significantly for either group. For those high in spiritual motivations, apprehension (β=−0.211) was a significant inverse predictor, whereas it had no significant predictive power for those low in spiritual motivations.

Predicting response Using the Ego Dissolution Inventory (EDI)

This model also explained a significant amount of variance in the EDI. In a backwards stepwise regression, the predictors explained nearly half of the variance in EDI (R²=0.497, Adj. R²=0.483), similar to the predictive level explaining mystical experience. The significant predictors in the best-fitting model predicting EDI included the set state of surrender ((3=.561, p<.001), deservingness (β=−0.210, p<0.001), absorption (β=0.162, p<0.05), and spiritual motivations (β=0.145, p<0.05).

Revised model predicting adverse experience

The initial model explored to predict adverse-DED used the same sequence of blocks and variables used to explore mystical-OBN in Table 8. Demographic factors in Block 1 (age, education, sex) explained 4.2% of the variance in adverse-DED (Adj. R²=0. 026). Trait variables in Block 2 (openness, deservingness, barriers, rigidity, absorption, and entitlement) increased the explanatory power to 11.1% (Adj. R²=0.065), and prior state variables (thriving, confusion) in Block 3 increased the explanatory power significantly to R²=0.252 (Adj. R²=0.204). The addition of motivations for having the psychedelic experience in Block 4 (spiritual, norms, and pleasure) and Setting variables in Block 5 (time with eyes closed, number of people present, amount preferred, and words) did not add significantly to the model. The addition of surrender, apprehension, and preoccupation in Block 6 significantly increased the predictive power to 39.5% of the variance in adverse-DED (Adj. R²=0.315).

All variables with Beta weights with p<0.1 in any block were retained for inclusion in a backwards stepwise regression predicting adverse-DED. These included age, absorption, openness, confusion, betterment motivations, pleasure motivations, apprehension, and preoccupation. The best-fitting model, shown in Table 8, included preoccupation (β=0.359, p<0.001), apprehension, (β=0.159, p=0.013), confusion (β=0.220, p=0.003), and openness (β=−0.129, p=0.040), and explained 34.4% of the variance in adverse-DED (Adj. R²=0.329). When compared for males and females, preoccupation and apprehension were both retained as significant predictors, but only age contributed additional significant (inverse) explanatory power for females, whereas openness and betterment motivations added explanatory power for males.

TABLE 8
Best-fitting model predicting adverse-DED
based on backwards stepwise regression

| | Unst. Coeff. | | Std. Coeff. | | | |
|---|---|---|---|---|---|---|
| Model | B | Std. Err. | Beta | t | Tolerance | VIF |
| (Constant) | .154 | .075 | | 2.041 | | |
| 2_Openness | −.030 | .014 | −.129 | −2.070* | .954 | 1.049 |
| 5_Confusion | .027 | .009 | .220 | 3.056** | .715 | 1.399 |
| 6_Apprehension | .031 | .013 | .159 | 2.517* | .925 | 1.081 |
| 6_Preoccupation | .057 | .012 | .359 | 4.949*** | .702 | 1.425 |

***p < .001;
**p < .01;
*p < .05.

Long-Term Response

Independent t-tests showed a strong and significant relation of mystical experience with long term outcomes. Mean scores for positive change among those with complete mystical experience were 4.00, significantly higher than the mean for those without such an experience, who were not far above the neutral point of 3=no change. For negative change, means for both group were in the neutral to decreased range, but scores for those with a complete mystical experience (M=2.22; t=5.837, p<0.001) indicated lower negative states than those without such an experience (M=2.75; t=−3.569, p<0.01). Those with a complete mystical experience rated their experience as far more important (M=75.66) than did those without such an experience (M=42.75; t=9.261, p<0.001). Finally, mean scores for flourishing were significantly higher for those with a complete mystical experience (M=4.78) than for those without (M=4.39; t=2.170, p<.05). See Table 9.

TABLE 9
Independent t-test of mean long term change scores
by complete or incomplete mystical experience.

| Scale | Mystical experience | N | Mean | Std. Deviation | Std. Error Mean | t-Test |
|---|---|---|---|---|---|---|
| Flourishing | None/partial | 129 | 4.39 | 1.03872 | .09145 | 2.170* |
| | Complete | 51 | 4.78 | 1.14350 | .16012 | |
| Importance | None/partial | 129 | 42.75 | 24.69703 | 2.17445 | 9.261*** |
| | Complete | 51 | 75.66 | 20.07324 | 2.81082 | |
| Negative | None/partial | 129 | 2.74 | .62887 | .05537 | −3.569** |
| Change | Complete | 51 | 2.22 | .95804 | .13415 | |
| Positive | None/partial | 129 | 3.39 | .54285 | .04780 | 4.909*** |
| Change | Complete | 51 | 4.00 | .82237 | .11515 | |

Scores for importance were based on a slider scale ranging from 1 to 100. Scores for positive change and negative change were based on a 5-point rating scale ranging from 1=Decreased significantly (3=“no change”) to 5=increased significantly. Flourishing was rated on a 6-point scale ranging from 1=strongly disagree to 6=strongly agree.

Mediating Role of Mystical Experience

To explore the plausibility of mystical-OBN as a mediator in predicting positive change, an initial set of predictors was determined by entering all of the trait variables into a linear regression model with positive change as a dependent measure, retaining the standardized predictors that predicted positive change with Beta weights p 21 0.1. The composite variable PC Traits included openness (β=0.154, p=0.097), absorption (B=076, p=0.004), and barriers (β=−0.139, p=0.087) was created based on the best-fitting regression model predicting positive change. PC Traits produced 6=.39 (p<0.001) in explaining positive change (FIGS. 2), and β=0.497 explaining mystical-OBN. Adding mystical-OBN, β=0.505 (p<0.001), with traits into a regression model predicting positive change reduced the coefficient for traits to 0.19 (p<0.05), while increasing explanatory power from 0.146 to 0.275 (Adj. R²), demonstrating plausibility of mystical-OBN as a meaningful partial mediator of the relations of trait variables with positive change. SEM analysis produced strong goodness of fit characteristics, including a nonsignificant chi square (x²=9.27, p=0.16), a RMSEA of 0.055, a normed fit index (NFI) of 0.997, a goodness of fit index of 0.984, and a comparative fit index of 0.999. A Sobel test of mediation produced a significant test statistic of 5.55 (SE=0.032, p<0.001).

Similar processes were utilized to predict the impact on other long-term measures. ME was found to have no significant value in predicting flourishing, for which the significant predictors included preoccupation (β=−0.416, p<.001), deservingness (β=−0.320, p=0.001), and spiritual motivations (β=0.222, p<.05) in a model explaining 25% of the variance (excluding prior state variables). Importance of the experience was predicted strongly by mystical-OBN (β=0.646, p<0.001), absorption (β=0.169, p<0.001), and rigidity (β=−0.197. p<.01) in a model explaining 52.5% of the variance. Relations of surrender with importance were rendered nonsignificant when mystical-OBN was entered into the model.

### Discussion

The first objective of this study was supported: Key predictors in the best-fitting regression models in Example 1 predicted similar variance in the current sample (S2) despite different measures of mystical and adverse experience. The regression model explained 66% of the variance in mystical-MEQ for S1 and 52% of the variance in mystical-OBN for S2, and the state and trait variables that were strongest in S1 were similar in strength in S2: surrender at the time of ingestion (β=0.607 in S1 and 0.525 in S2), and the trait absorption (β=0.308 in S1 and 0.333 in S2). Likewise, the best-fitting model predicting adverse experience (dread) in S1 explained similar proportions of variance in adverse-DED in S2 with similar Beta weights among the strongest predictors: preoccupation followed by confusion as an inverse predictor. Variables with smaller Beta weights in S1 did not contribute significantly in S2 for mystical or adverse experience. This is a notable replication of the basic predictive models for mystical and adverse experience because (a) the surrender and preoccupied scales appeared to be robust constructs with good internal consistency and loadings in two samples, (b) the robust constructs were retained as the strongest predictors of response to psilocybin in a second sample, (c) the significant inverse predictive power of words (the extent to which conversations and words were present during the psilocybin session) for mystical experience in both samples supports the control given to the auditory stimuli in therapeutic settings, and (d) the model predicted strongly with different measures of mystical experience and dread (the 5D-ASC) which, although correlated strongly with the MEQ-30 (Leichti, et al., Psychopharmacology 2017, 234:1499-1510), cannot be presumed identical although isomorphic.

Additional hypothesized predictors were also examined in a second hierarchical regression in S2. The key predictors were the same: surrender (β=0.453, p<0.001) and absorption (β=0.247, p<0.001) remained the strongest predictors of mystical-OBN in S2 just as in S1, but spiritual motivations for taking psilocybin positively predicted mystical-OBN (β=0.219, p<0.001), and level of apprehension (β=117, p<0.05) and a trait of deservingness (β=−0.162, p<0.01) predicted inversely. Although multicollinearity was not a problem in the model (VIF=1.519), spiritual motivations correlated significantly with absorption (r=0.53, p<0.01) and with surrender (r=0.40, p<0.01), suggesting that some people may be more dispositionally oriented toward spirituality, which in turn might facilitate a greater level of surrender and stronger mystical experience.

To explore the possibility that spiritual motivations may have promoted more mystical interpretations of the experience, the best-fitting model predicting mystical-OBN was generated separately for those above and below the mean in spiritual motivations. The best-fitting models were similar for both groups, with surrender as the strongest predictor, and absorption and deservingness (inverse) as significant trait predictors; only apprehension served as a unique inverse predictor for those higher in spiritual motivations. If it can be presumed that the OBN subscales of the 5D-ASC measures a “mystical” construct similar to the MEQ-30, as is suggested by correlations >0.8 between the two measures (e.g., Leichti, et al., Psychopharmacology 2017, 234:1499-1510), then the current model suggests that it is the state of surrender in conjunction with particular traits rather than spiritual motivations that contribute most significantly to mystical experience.

For explaining adverse-DED, preoccupation had the strongest Beta weight, but confusion and apprehension also predicted positively and significantly (p<0.01) and openness predicted inversely. In this sample, openness was strongly related to absorption (r=0.51, −<0.01) and inversely related to rigidity (r=−0.28, p<0.01), converging with other scales as expected.

The current study suggests a mediating role for mystical experience in promoting positive long term change in personal meaning and spirituality. An independent t-test compared several measures of current state or change between those who had a complete mystical experience and those who did not, according to the threshold established by Barret, et al. (Journal of Psychopharmacology 2015, 29:1182-1190). Having a complete mystical experience was associated with significantly higher scores in positive change (e.g., gratitude, joy, trust, empathy, and social concern) and significantly lower scores in negative change (e.g., anxiety, fear, impatience) than reported by those with sub-threshold levels of mystical experience. The current study also suggests that mystical experience also mediated the relationship between trait predictors and positive change.

### Example 3. Predictors of Meditation-Induced Mystical Experience and Positive Response

The following study was performed to develop and characterize a set of mental state and trait predictors of mystical experience (ME) and to identify predictors of positive response to intensive meditation.

### Methods

Participants

Participants were selected from a U.S. population who had participated in an intensive meditation retreat of three or more days in the past year. Participants were recruited through Amazon Mechanical Turk (MTurk), an online source in which individuals participate in survey studies for small cash compensation. The final screened sample consisted of 110 valid responses with 48 males (43.6%) and 62 females (56.4%), and an average age of 31.8 (SD=9.91) ranging from 19 to 61 years of age. Twenty-one percent of participants had a graduate degree or some graduate education, 40% had completed a Bachelor Degree, 20% reported that they had some college, 14% had completed an Associate Degree, and 5% had not yet continued their education beyond a high school diploma.

60% of respondents reported that formal religion had been important or very important in their childhood families, but only 42% reported that it was still important or very important in their lives today;

in fact, 39% reported that formal religion was not at all important in their lives today. In contrast, 80% reported that spirituality was important or very important in their lives today, and only 8% reported that spirituality was not at all important. 31% of participants reported that they meditate at least once per day, 26% reported that they meditate one or several times per week, and 29% reported that they pray one or more times per day. 51% of participants reported that they practice yoga once or more per week.

56% of participants reported that they had never used a psychedelic drug. Of those who reported they had used psychedelic drugs, 59% reported that the first psychedelic ingestion preceded their first meditation experience. About 66% of participants reported that they never used marijuana or used it not more than once per year, compared with 13% who reported at least daily use of marijuana.
Materials

The online survey consisted of 290 items on an online survey tool Survey Monkey. Participants were directed to the relevant webpage after reading informed consent text, completing screening and agreeing to participate in accordance with the stipulations of the Institutional Review Board. Scaled items in all but the long-term outcomes used four-point Likert scales ranging from 1=Strongly Disagree to 4=Strongly Agree. Answers were required for each item, but each included an option “I prefer not to answer.” Open-ended questions designed to activate participants' memories of the retreat were completed before responding to scale items related to crisis and mystical experience. On the closing page, participants were asked to indicate on a four-point scale how carefully and accurately they had been able to respond to the questions, and were told that answers would not affect financial compensation of $1.00.

Background Items

Background items requested information about participants' sex, education, age, and importance of formal religion and spirituality in their childhood family and in their lives today.

Trait Measures

Items from a variety of trait scales were intermixed randomly and presented in blocks of five statements each, along with four attention items written to be semantically similar to the surrounding items.

These scales included:

Tellegen Absorption Scale (TAS)

The 34-item TAS assesses a trait or disposition “for having episodes of ‘total’ attention that fully engage one's representational (i.e., perceptual, enactive, imaginative, and ideational) resources” originally developed to predict hypnotizability (Tellegen and Atkinson, Journal of Abnormal Psychology 1974 83(3), 268-277). In this sample, Cronbach's alpha α=0.929. The TAS has been found to predict ME-related phenomena in psychedelic sessions (Studerus et al., PLoS ONE2012, 7).

Mental Barriers scale

This newly developed six-item, two-component scale assesses the tendency to reject certain types of information or experiences. The scale is based on descriptions provided by James (The varieties of religious experience: A study in human nature, Longmans, Green and Co. New York, N.Y., 1902) and Abraham Maslow (Journal of Transpersonal Psychology 1970, 2(2):83-90), who described the hindering capacity of such barriers. Using a principal components analysis (PCA), the six items in the final scale produced a simple two-factor structure explaining 68.7% of variance with Varimax rotated loadings ranging from 0.755 to 0.835 on Component 1 and −0.864 and −0.872 on Component 2 and internal consistency (Cronbach's Alpha) α=0.737. The first component included four items:

(i) I reject ideas that can't be logically explained;

(ii) I reject ideas that are not supported by experts in the field;

(iii) I reject ideas that are based solely on others' personal experiences; and

(iv) I only accept ideas that have scientific evidence behind them;
and the second component included two reverse items:

(i) I have had experiences of knowing something without knowing how I knew it; and (ii) My intuition has helped me at times.

Meditation History

This section asked about meditation experiences prior to the retreat they attended, and included questions about how long they had meditated, how consistent their meditation practice was, how often they practiced mindfulness techniques, prayer, meditation, yoga, and used marijuana, and whether they had used psychedelic drugs at any point.

Days and Weeks Preceding the Meditation Retreat

These items examined the events and emotions in participants' lives in the days and weeks before the retreat to identify the extent of identity distress. Initially, participants' memory of this period was activated through questions such as “Please describe what was happening in your life in the days and weeks immediately preceding your meditation experience.” Next, participants completed 28 Likert-scaled items measuring psychological state in the days and weeks before the experience. This measured three a priori dimensions, of which only one was used within the model due to problems with collinearity. That variable was distress, measured by five items that appeared to capture the state of turmoil associated with personal change (see James (The varieties of religious experience: A study in human nature, Longmans, Green and Co. New York, N.Y., 1902). The items that loaded into a single factor in this sample included: “I'd no longer had a sense of who I was,” and “I'd felt that my identity was changing.” The final internally consistent measure (α=0.856) explained 63% of the variance in the items.

Scale items were intermixed in this section and also included three attention items (e.g., “I will mark three for this item”) to identify and exclude data from inattentive participants.

Entering the Retreat

Individuals' mindsets we assessed as they entered the retreat with the instruction: “The next items relate to your mindset as you entered the meditation experience. It may be unchanged from the days before the experience, but perhaps your emotions shifted when you first entered the meditation experience. This is the time in which we are interested. Thank you.” The items contained 24 statements related to their mental state and one item designed to detect inattentive participants (“Four is the correct response for this item”). All items were intermixed in five-item blocks and rated on a four-point scale. The final scales, “State of Surrender (SoS)” and “Preoccupation” were preliminarily validated in a larger sample. The loadings for this sample are presented below.

State of Surrender (SoS) Scale

The final scale included an average score of ten statements related to a state of surrender, or a readiness to accept whatever was, whether good or bad, without resisting or fighting or struggling. Items were created based on James's descriptions and validated them in two separate samples. Using PCA, a single factor was identified with ten items explaining 54.5% of variance among the items with loadings ranging from 0.705 to 0.778 with internal consistency using Cronbach's Alpha α=90.07. A check for internal consistency within the sample used for this study found Cronbach's Alpha α=0.894. Sample items included “I had stopped resisting and was ready to give up control,” and “I'd felt a release from the need to think so hard.” The scale items were identified using PCA on a normed sample (n=232) consisting of the individuals in this sample and a second sample who had taken psilocybin.

Preoccupation Scale

The final Preoccupation scale included an average score for four items related to preoccupation with one's immediate life (e.g., “I felt a little bit rushed for time” or “I had a lot of things on my mind at the time”). A PCA was conducted on the norming sample (n=232) that included both this sample and a second sample of individuals who reported they used psilocybin. The PCA met all assumptions and produced loadings ranging from 0.711 to 0.814 with an Eigenvalue of 2.269 explaining 56.7% of the variance and internal consistency of α=0.745. Internal consistency within this sample was Cronbach's Alpha α=0.704.

Details of the Meditation

Open-ended and multiple choice questions were used to gather information about the type of retreat, their reasons for attending the retreat, how they prepared for the retreat, how much talking or reading was allowed, how many sits they attended, and how much time was occupied in each sit.

During the Most Memorable, Intense, or Meaningful Sit

We measured participants' subjective experience during the most memorable, intense, or meaningful sit. We first asked participants to “identify one session you would describe as particularly meaningful, intense, and memorable” and describe this session in an open-ended question. We next asked participants to respond to scale items based on the memorable sit described on the preceding page. The scale items contained 70 intermixed items on a four-point Likert scale including 30 items from the Mystical Experience Questionnaire (MEQ30; MacLean et al., Journal of the Scientific Study of Religion 2012, 51(4):721-737),36 items from the Sacred Emotions Scale (SES; Burdzy, Sacred Emotions Scale (Thesis; 2014), Bowling Green State University, Kentucky), and four items to detect inattention.

MEQ30

The MEQ30 is a validated scale consisting of 30 items utilized in psychedelic studies (Barrett et al., Journal of Psychopharmacology 2015, 29(11):1182-1190; MacLean et al., Journal of the Scientific Study of Religion 2012, 51(4):721-737). Some of the items from the MEQ30 were adapted for this study so that they were semantically consistent with the other scale (e.g., changed “I felt certain that I'd encountered ultimate reality” to “Certainty that I'd encountered ultimate reality”), and used a four-point rather than a seven-point scale for reasons described above. The scale measures four dimensions of a mystical experience: (a) Mystical; 15 items with α=0.950 in this sample, (b) Ineffability; 3 items with α=0.742 in this sample, (c) Timelessness/Spacelessness; 6 items with α=0.845 in this sample, and (d) Positive

Affect; 6 items with α=0.862 in this sample. The total scale produced Cronbach α=0.965.

DREAD-Sacred Emotions Scale (SES)

Burdzy and Pargament developed and validated the 41-item SES. The SES measures the “emotional impact of an individual's experience with the sacred” (p. iii). Only 36 items were included because a section containing one set of five items was unintentionally deleted. Because the exuberance scale was strongly correlated with the MEQ30 (Pearson r=0.803), it was not used in any of the analyses. The twelve items used were specifically related to dread, including feelings of powerlessness (e.g., I felt completely insignificant,” “I felt small”), worry (e.g., “I felt afraid,” “I felt ashamed”), and behavioral representations of these feelings (e.g., [I felt like] turning away”). This sample produced internal consistency scores of (Cronbach's alpha) α=0.880.

After the Retreat

Participants were asked to describe their current state in three pages. First, they were asked whether they would attend another intensive retreat if they had a chance. Forty-six statements followed, including 15 intermixed items adapted from Griffiths et al., (Psychopharmacology 2006, 187:268-283) measuring Positive Emotions, Negative Emotions, and Spirituality, to which subjects responded on five-point Likert scales ranging from “Strongly Decreased” to “Strongly Increased.” A PCA was conducted on a larger sample (n=232) that included this sample and a separate sample of individuals who had used psilocybin. After four items were eliminated, a two-factor simple structure emerged that explained 63.8% of the variance (Eigenvalue=5.740), and internal consistency (Cronbach's Alpha) α=0.929. Items included “creativity and playfulness,” “gratitude and contentment,” “joy and optimism,” “trust that everything will work out,” “social concern and compassion,” “empathy and sensitivity to others,” “honesty and authenticity,” “patience and tolerance,” and “confidence and assurance.” Items on the second component were reversed and a mean was calculated to create the variable Positive Change. Internal consistency for this sample alone was (Cronbach's Alpha) α=0.907.

### Procedures

Data Collection

Data were gathered from Amazon Mechanical Turk (MTurk), an online crowdsourcing tool through which participants can choose from among a wide variety of surveys for small monetary compensation. MTurk broadly represents the general population than the undergraduate population often used in psychological studies and provides findings equivalent to studies conducted in laboratories. Compensation was $1.00.

Recruitment

The original MTurk post sought 150 residents of the United States who had participated in a recent intensive meditation retreat and were willing to respond to a lengthy survey taking approximately 40 minutes to complete with more than 250 questions about their experience. Definitions of “intensive” and “recent” were not provided so that individuals would not adjust their responses to gain admittance to the survey, such as describing their retreat as a three-day retreat when in actuality it was a two-day retreat. If they passed screening items, they were given a link and password to the informed consent followed by the survey.

Screening

A total of 979 individuals entered the screening page and responded affirmatively to the question, “Are you 18 years of age or older?” A total of 546 individuals affirmed that they attended an intensive meditation retreat in 2015 or 2016, and 259 individuals indicated they participated three or more days. Of the 231 individuals who indicated that they had 35-45 minutes to donate, 221 provided their informed consent, 177 individuals entered the survey, and 144 completed the majority of the survey.

Validation

The final screening process eliminated participants who did not: (a) respond accurately to more than 9 of the 12 attention items, and (b) indicate that they had responded carefully to all or most of the items by selecting ≥3 on the accuracy question at the end of the survey. This step eliminated 24% (n=34) of those who had entered the survey, including six of seven participants who responded correctly to ≥9 attention items but had missing values for the accuracy item, yielding a final sample of N=110. The mean number of correct attention items in the final sample was 11.4 of 12 (SD=0.94), and the mean for the non-selected group was 6.7 (SD=2.93).

Statistical Approach

Hierarchical linear multiple regression with data entered in blocks was used to (1) determine the explanatory power of hypothesized variables on dependent measures of ME and Dread, and (2) identify a potentially mediating role of ME on long-term positive change. In all cases, blocks were entered into the model in logical order of their appearance in and probable influence on an individual's life. Block 1 included “Demographic” factors (Age, Sex, and Education). Block 2 included “Religion” (importance of family religion and current religion). Block 3 included “Habits” (frequency of marijuana use, meditation, yoga, and mindful practices). Block 4 included “Trait and Belief” factors (TAS, mental barriers). Block 5 included “Prior State” conditions in the days and weeks before the retreat (vulnerability, distress, and emotionality). Block 6 included “Retreat” factors (amount of talking/reading, number and length of sits). Block 7 included “State” factors experienced at the start of the most memorable sit (surrender and preoccupation). Predictors were narrowed through two processes: (1) eliminating blocks that did not present any significant changes to R², and (2) eliminating predictors within blocks if collinearity was present.

Each model was then examined for collinearity problems or other violations of the assumptions for regression. Multicollinearity was discovered among three predictor variables (exposure, vulnerability, and distress) measuring mental state in the days and weeks before the meditation, which had Pearson r-values in excess of 0.7 and tolerance values <0.3. The strongest predictor, distress, was therefore retained in the model. Following the elimination of the other variables, all of the observations were independent with acceptable levels of auto-correlation in the residuals (Durbin-Watson statistic=2.2). Because individuals were offered the option of “I prefer not to answer” for every scale item, there were occasional missing data points. Respondents with one or more missing values across items ranged from 2% of participants (i.e., distress, preoccupation) and 10% (i.e., state of surrender) of participants. Mean scores for the scales were calculated without the missing data points for individuals who did not respond to a given item, and they were included in the regression analysis. Outliers were identified as those whose residuals were ≥3 standard deviations from the predicted value. These were examined individually to determine if there was an unusual pattern present, and eliminated or retained based on that evaluation.

### Results

Correlations among new and previously validated scales

Because new constructs were evaluated, a correlation matrix of all the variables measured were examined to assess their construct validity (Table 10). Several interesting relationships may be noted.

First, absorption correlated positively with emotionality (r=0.473**), distress (r=0.359**), state of surrender (r=0.428**), and the MEQ (r=0570**). Mental Barriers corresponded inversely with emotionality (r=-0.216*), SoS (r=−0.232*), and the MEQ (r=−0.271 **), and correlated positively with Preoccupation (r=0.223**). Meditation frequency corresponded positively with absorption and MEQ (r=0.283** and 0.250**, respectively), while marijuana frequency corresponded positively with distress, emotionality, and exposure (r=0.200*, 0.230*, 0.240*, respectively) and also with absorption (r=0.266**). Distress corresponded positively with preoccupation and dread (r=0.504** and .358**, respectively) while emotionality corresponded positively with ME (r=0.412**), state of surrender (r=0.382**), and preoccupation (r=0.353**). Finally, state of surrender corresponded positively with ME (r=0.653**), and preoccupation corresponded positively with both dread and MEQ (r=0.415** and 0.210*, respectively). Two-tailed tests of significance were used throughout.

TABLE 10
Correlations among predictor and dependent measures.

| | Age | Edu | Sex | TAS | MB | RelF | RelF | MedF |
|---|---|---|---|---|---|---|---|---|
| Age | 1 | | | | | | | |
| Edu | .257** | 1 | | | | | | |
| Sex | .113 | −.089 | 1 | | | | | |
| TAS | −.124 | −.035 | −.068 | 1 | | | | |
| MB | −.053 | −.011 | .373** | −.169 | 1 | | | |
| RelF | .158 | .136 | −.248** | .107 | −.135 | 1 | | |
| RelT | .250** | −.015 | −.090 | −.101 | .082 | .498** | 1 | |
| MedF | −.020 | −.006 | .066 | .283** | −.080 | −.095 | −.078 | 1 |
| MarF | −.296** | −.286** | .075 | .266** | .097 | −.161 | −.220* | .136 |
| Dis | −.250** | −.203* | −.038 | .359** | .100 | .020 | .037 | .135 |
| Emo | −.333** | −.124 | −.204* | .473** | −.216* | .046 | −.074 | .163 |
| Exp | −.242* | −.175 | −.054 | .280** | −.055 | −.115 | −.172 | .128 |
| Pre | −.120 | −.019 | .161 | .306** | .223* | .057 | .076 | .036 |
| SoS | .023 | −.005 | −.067 | .428** | −.232* | .081 | −.003 | .172 |
| Dread | −.072 | −.025 | .258** | .101 | .425** | .027 | .106 | −.039 |
| ME | .063 | .028 | .009 | .570** | −.271** | .150 | .044 | .250** |
| | MarF | Dis | Emo | Exp | Pre | SoS | Dread | ME |
| Age | | | | | | | | |
| Edu | | | | | | | | |
| Sex | | | | | | | | |
| TAS | | | | | | | | |
| MB | | | | | | | | |
| RelF | | | | | | | | |
| RelT | | | | | | | | |
| MedF | | | | | | | | |
| MarF | 1 | | | | | | | |
| Dis | .200* | 1 | | | | | | |
| Emo | .230* | .541** | 1 | | | | | |
| Exp | .240* | .656** | .697** | 1 | | | | |
| Pre | −.018 | .504** | .353** | .393** | 1 | | | |
| SoS | .038 | .169 | .382** | .231* | .131 | 1 | | |
| Dread | .118 | .358** | .011 | .104 | .415** | .031 | 1 | . |
| ME | .136 | .146 | .412** | .233* | .210** | .653** | .064 | 1 |

Edu = Education; TAS = Tellegen Absorption Scale, MB = Mental Barriers, RelF = Importance of religion in childhood family; RelT = Importance of religion today; MedF = Frequency of meditation; MarF = Frequency of marijuana use; Dis = distress; Emo = Emotional; Exp = Exposure; Pre = Preoccupation; SoS = State of Surrender; MEQ = Mystical Experiences Questionnaire- 30 item version.

Predicting ME

A preliminary regression model showed that demographic factors in Block 1 produced R2=0.001 which, when adjusted for the number of variables, explained none of the variance in ME (F=0.046, ns), and the addition of “Religion” elements in Block 2 added nothing to the model (F=0.700, ns). With the addition of “Habits” in Block 3, the model strength increased significantly (F=2.078*) to explain 8.9% of the variance in ME with the strongest predictor Frequency of Meditation (Beta=0.334**) and Importance of religion in childhood family (Beta=0.234*). Adding “Traits and Beliefs” to the model in Block 4 significantly expanded the explanatory model (F=6.321***) to explain 37.2% of the variance through Absorption (Beta=0.517***) and barriers (Beta=−0.250) only. The addition of “Prior State” factors in Block 5 increased the explanatory power of the model significantly but also added multicollinearity with tolerance values <0.425 among these predictors. The addition of “Retreat” factors in Block 6 added significantly to the model to explain a total of 41.6% of variance, but none of the retreat factors were predictive in this model and collinearity among the “prior state” variables persisted. Finally, the addition of “State” factors increased the explanatory power to 49.4% explained in the model by state of surrender (Beta=367) and absorption (Beta=0.283**). According to the protocol, the model was refit to eliminate items producing multicollinearity, non-hypothesized predictors, and nonsignificant predictors. A second regression model was fit.

The final regression model included: Block 1: Absorption; Block 2: Mental Barriers; Block 3: a prior state of Distress; Block 3: Surrender and Preoccupation at the start of the retreat. One outlier was present with a predicted value more than three standard deviations from what was expected. No anomalous patterns were detected in the outlier, so the case was retained within the model. As is shown in Table 11, the overall regression model explained 50.3% of the variance in ME. Model 1 shows that absorption alone explained 32% of the variance in ME. The explanatory power increased slightly but significantly (F=28.83***) to 34% with the addition of mental barriers in Model 2. The addition of distress in Model 3 did not increase the explanatory power of the model. With the addition of preoccupation and state of surrender in Model 4, the explanatory power increased significantly to explain a total of 50.3% (adjusted R²) of the variance in ME. In this final model, state of surrender and absorption were significant predictors with Beta weights of 0.469 and 0.305, respectively. Mental barriers (β=−0.124), distress (β=−0.100), and a state of preoccupation at the start of the sit (β=0.128) were not significant predictors.

TABLE 11
Linear Regression Predicting ME with Trait and State Factors

| | | B | SE | β | t | Sig. | Adj. R² |
|---|---|---|---|---|---|---|---|
| Model 1 | (Constant) | .676 | .324 | | 2.087 | .039 | .318 |
| | Absorption | .792 | .108 | .587 | 7.316 | .000 | |
| Model 2 | (Constant) | 1.569 | .456 | | 3.441 | .001 | .340 |
| | Absorption | .677 | .113 | .502 | 5.980 | .000 | |
| | Mental barriers | −.271 | .100 | −.227 | −2.703 | .008 | |
| Model 3 | (Constant) | 1.599 | .459 | | 3.486 | .001 | .334 |
| | Absorption | .706 | .120 | .523 | 5.903 | .000 | |
| | Mental barriers | −.266 | .101 | −.223 | −2.644 | .010 | |
| | Distress | −.048 | .064 | −.062 | −.754 | .452 | |
| Model 3 | (Constant) | .372 | .380 | | .979 | .330 | .503 |
| | Absorption | .423 | .116 | .305 | 3.638 | .000 | |
| | Mental barriers | −.139 | .082 | −.124 | −1.694 | .093 | |
| | Distress | −.078 | .063 | −.100 | −1.240 | .218 | |
| | Preoccupation | .121 | .077 | .128 | 1.573 | .119 | |
| | Surrender | .527 | .090 | .469 | 5.863 | .000 | |

Dependent Variable: Mystical Experience (ME)

Dread

Table 12 displays data for a regression model with the same variables used to predict dread.

The trait variable absorption explained no variance, as shown in Block 1, and when combined with mental barriers explained 20% of variance in Block 2 (F=14.5***). The addition of distress in Block 3 increased the explanatory power to 26.7%, and the addition of preoccupation and surrender variables in Block 4 added even more to prediction to explain a total of 29.1% (adjusted R²) of the variance in dread. In the final model, mental barriers, preoccupation and distress were the strongest predictors of a dread experience with Beta weights of 0.368, 0.225, and 0.197, respectively. Neither absorption nor state of surrender were influential in predicting dread.

TABLE 12
Linear Regression Predicting Dread with Trait and State Factors

| | | B | SE | β | t | Sig. | Adj. R2 |
|---|---|---|---|---|---|---|---|
| Model 1 | (Constant) | 1.510 | .385 | | 3.926 | .000 | .001 |
| | Absorption | .133 | .127 | .101 | 1.045 | .298 | |
| Model 2 | (Constant) | .148 | .431 | | .343 | .732 | 200 |
| | Absorption | .249 | .116 | .188 | 2.145 | .034 | |
| | Mental barriers | .491 | .093 | .461 | 5.257 | .000 | |
| Model 3 | (Constant) | .116 | .412 | | .281 | .779 | .267 |
| | Absorption | .106 | .119 | .080 | .893 | .374 | |
| | Mental barriers | .440 | .091 | .412 | 4.841 | .000 | |
| | Distress | .217 | .066 | .291 | 3.281 | .001 | |
| Model 3 | (Constant) | .019 | .433 | | .044 | .965 | .291 |
| | Absorption | .033 | .133 | .025 | .249 | .804 | |
| | Mental barriers | .393 | .093 | .368 | 4.203 | .000 | |
| | Distress | .147 | .072 | .197 | 2.053 | .043 | |
| | Preoccupation | .203 | .088 | .225 | 2.311 | .023 | |
| | Surrender | .032 | .103 | .030 | .315 | .753 | |

Dependent Variable: Dread
Prior Distress as Moderator

Table 13 shows that the regression model predicted mystical experience differently for people with high and low distress in the days and weeks preceding the meditation retreat. The threshold for high distress was set at a mean score on distress items ≥3. For people with high distress, the model explained 40.8% of the variance, with trait of absorption as the only significant predictor (β=0.492). For those with low distress, the model predicted 59.3% of the variance in mystical experience, attributed to the state of surrender (β=0.639, p<0.0001) and preoccupation (β=0.168, p<0.05).

TABLE 13
Linear regression model for MEW for individuals
with high and low distress prior to the retreat.

| | |
|---|---|
| Low Distress | High Distress |
| β (t-value) | β (t-value) |

| | | | | |
|---|---|---|---|---|
| (Constant) | .163 | (1.795) | .492 | (3.359**) |
| Absorption | | | | |
| Mental barriers | −.034 | (−.405) | −.155 | (−1.132) |
| Preoccupation | .168 | (2.039*) | −.042 | (−.325) |
| Surrender | .639 | (6.909***) | .207 | (1.517) |

| | | |
|---|---|---|
| Adj. R² | .593 | .408 |

*p < 0.05;
**p < 0.01;
***p < 0.001

For predicting dread, the model also differed between those with high and low distress, as shown in Table 14. For those with distress levels ≥3, the model explained 31% of the variance in dread, attributable primarily to mental barriers (Beta=0.633***), but for those with lower distress the model explained only 18.5% of variance most significant predictor was a state of preoccupation with a Beta weight of 0.408. None of the other variables were significant predictors for those with high or low distress.

TABLE 14
Linear regression model predicting dread for
individuals with high and low distress

| | |
|---|---|
| Low Distress | High Distress |
| β (t-value) | β (t-value) |

| | | |
|---|---|---|
| (Constant) | (1.211) | (.007) |

| | | | | |
|---|---|---|---|---|
| Absorption | .002 | (.014) | .161 | (1.022) |
| Mental barriers | .195 | (1.643) | .633 | (4.285***) |
| Preoccupation | .408 | (3.508**) | .038 | (.271) |
| Surrender | .014 | (.106) | −.064 | (−.433) |

| | | |
|---|---|---|
| Adj. R² | .185 | .313 |

**p < .01;
***p < .0001

Positive Change

The regression model to predict positive change was created using the same predictors as for ME, but adding ME itself in a separate block to explore its impact as on positive change. ME was entered as a fifth block predictor within the regression model in order to test the hypothesis that ME mediates the relationship between other predictors and positive change. The mean positive change score was 4.01 (SD=0.68), with higher scores representing greater increases in positive emotions. Tolerance statistics suggest a potential problem with collinearity, with variance inflation (VIF) factors for ME (VIF=2.168) slightly exceeding optimal levels. However, because it was hypothesized that a mystical state mediated the relationship between the predictors and positive change and the collinearity level was known from the ME regression reported above, the model was retained.

As is shown in Table 15, the variables that were originally used to predict ME explained 19% of the variance in positive change, but when ME was added to the model as a predictor, the explanatory power increased significantly to 25.9%. Before adding ME, absorption (β=−0.219), and surrender (β=0.267) were both significant predictors in the best-fitting model of positive change. When ME was added to the model, ME had the largest and only significant Beta weight (β=0.325) eliminating the significance of every other predictor in the model.

TABLE 15
Linear regression model for positive change

| | Model 1 (without ME) | | Model 2 (with ME) | |
|---|---|---|---|---|
| | β (t-value) | | β (t-value) | |
| (Constant) | .219 | (2.04*) | .099 | (0.91) |
| Absorption | | | | |
| Mental barriers | −.157 | (−1.67)* | −.108 | (−1.19) |
| Identity Distress | −.027 | (−0.26) | −.013 | (−0.13) |
| Preoccupation | .076 | (.728) | −.126 | (−0.90) |
| Surrender | .267 | (2.61*) | −.083 | (−1.25) |

| | | | |
|---|---|---|---|
| Mystical Experience | — | .392 | (3.25)** |

| | | |
|---|---|---|
| Adj. R² | .190 | .259 |

*p < .05;
**p < .01

### Discussion

The final regression model predicted more than half the variance in ME. The most pivotal factor in predicting ME was a state of surrender, or a readiness to give in to whatever the experience offered, whether good or bad. When entered in a first block, the trait of absorption appeared to facilitate ME, and mental barriers decreased the likelihood of ME. Both of these variables combined explained 38.9% of the variance in ME, which increased significantly when SOS and Preoccupation were added to the model; its explanatory power increased to 50.3% of the variance in ME. A state of crisis prior to the retreat did not add explanatory power to the model.

Neither a state of surrender nor the trait of absorption played a significant explanatory role in predicting dread. Rather, it appeared that a prior state of distress in combination with high trait absorption and a state of preoccupation combined to explain 29% of the variance in the isolating experience of dread.

Although a prior state of distress did moderate the relationship between the predictors and the dependent measures, as shown in Tables 4 and 5, it did not align with surrender in increasing the likelihood of ME. For individuals high in prior distress, only absorption was a significant predictor of ME, explaining 40% of the variance in mystical experience. This finding compares to a robust predictive role of surrender in predicting 59% of the variance ME for those low in distress. None of the other variables predicted ME significantly for either group.

Distress also moderated the relationship between the predictors and dread. For those high in prior distress, the model explained 31% of the variance in dread, primarily attributable to the presence of mental barriers. No such relationship was found for those low in distress, for which only preoccupation weakly predicted an experience of dread.

Finally, the model produced some evidence to illustrate the mediating role of ME in predicting positive emotional change. A regression model including absorption, mental barriers, distress, surrender, and preoccupied alone predicted 19% of the variance of positive emotional change before ME was added to the model, primarily attributed to mental barriers (inversely) and state of surrender. After ME was added, the model explained 26% of the variance with all of the predictive explanatory power attributable to ME and no other predictors retaining significant Beta weights.

Rates of MEs found during psilocybin sessions vary substantially from 30-40% (Pahnke, International Psychiatry Clinics 1963, 5:149-162) to 60-72% (Griffiths et al., Psychopharmacology 2006, 187:268-283; Griffiths et al, Psychopharmacology 2011, 218:649-665). Using the MEQ30 with a cut point of >2.5 of 4 on all subscales, for example, 58% of psilocybin participants had a complete mystical experience, and a reanalysis with the cut point of 3 (used in the study provided herein) revealed that 45% of psilocybin participants reported complete MEs. High surrender and low mental barriers predicted mystical experience, facilitated by the trait of absorption. The capacity to bend one's entrenched beliefs, to cease resistance, to release one's time- and mind-based concerns and flex with whatever offers itself appears to smooth the pathway to ME.

The dread experience of isolation, fear, and emptiness experienced by some meditators appears to be partially explained by the same mechanism in reverse. Preoccupation with time and tasks were found in this study to predict 28% of adverse “dread” experiences in intensive meditation. One might posit that this state of preoccupation solidifies the default mode network so that neither meditation nor psychedelic drugs can penetrate the cycle of past/future/self-concerns.

Our study suggests that ME is associated with positive therapeutic response. The variance in positive outcomes accounted for by MEs reduced the explanatory power of every other variable and suggests that the ME itself has at least some role in producing positive change.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of improving mental or physical well-being of a subject, the method comprising:
(i) providing a subject, wherein based on a score of one or more predictors in the subject, the subject has been identified as likely to have a positive therapeutic response to a psychedelic agent; and
(ii) following step (i), administering to the subject the psychedelic agent.

2. The method of claim 1, wherein the method is for treating stress in the subject, treating anxiety in the subject, treating addiction in the subject, treating depression in the subject, or treating a compulsive behavior in the subject.

3. The method of claim 1 or 2, wherein the subject is obese, and the method comprises promoting weight loss in the subject.

4. The method of claim 1, wherein the method is for treating a condition in a subject, improving the mood of a subject, or enhancing the performance of a subject.

5. The method of claim 4, wherein the condition is a psychological disorder.

6. The method of any one of claims 1-5, wherein the one or more predictors comprises one or more trait predictors.

7. The method of claim 6, wherein the one or more trait predictors comprises a measure of absorption and/or a measure of mental barriers.

8. The method of claim 7, wherein a high measure of absorption is positively correlated with a positive therapeutic response.

9. The method of claim 7 or 8, wherein a high measure of mental barriers is inversely correlated with a positive therapeutic response.

10. The method of any one of claims 1-9, wherein the one or more predictors comprises one or more state predictors.

11. The method of claim 10, wherein the one or more state predictors are selected from the group consisting of a measure of distress, a measure of preoccupation, and a measure of surrender.

12. The method of claim 11, wherein a high measure of distress is positively correlated with a positive therapeutic response.

13. The method of claim 11 or 12, wherein a high measure of preoccupation is inversely correlated with a positive therapeutic response.

14. The method of any one of claims 11-13, wherein a high measure of surrender is positively correlated with a positive therapeutic response.

15. The method of any one of claims 1-14, wherein the one or more predictors comprises one or more habits.

16. The method of claim 15, wherein the one or more habits comprise frequency of meditation.

17. The method of claim 16, wherein the frequency of meditation is positively correlated with a positive therapeutic response.

18. The method of claim 15, wherein the one or more habits comprise frequency of cannabis use.

19. The method of claim 18, wherein the frequency of cannabis use is positively correlated with a positive therapeutic response.

20. The method of claim 1 or 2, wherein the one or more predictors are selected from the group consisting of a measure of absorption, a measure of mental barriers, a measure of meditation frequency, a measure of distress, a measure of preoccupation, and a measure of surrender.

21. The method of any one of claims 1-20, wherein the score is a composite score of at least two, at least three, at least four, at least five, or at least six of the predictors.

22. The method of claim 21, wherein the composite score is a weighted composite score.

23. The method of claim 22, wherein each predictor is weighted based on its explanatory power on positive therapeutic response to the psychedelic agent.

24. The method of claim 23, wherein the explanatory power is determined by a regression model.

25. The method of claim 24, wherein the factor analysis is a hierarchical linear multiple regression.

26. A method of improving the mental or physical well-being of a subject, the method comprising:
(i) providing a subject, wherein the subject has been identified as likely to have a positive therapeutic response to a psychedelic agent based on a high measure of:
absorption;
identity distress; or
surrender; and
(ii) following step (i), administering to the subject the psychedelic agent.

27. A method of reducing the risk of developing a psychological disorder in a subject in need thereof, the method comprising:
(i) providing a subject, wherein the subject has been identified as likely to have a positive therapeutic response to a psychedelic agent based on a high measure of:
absorption,
identity distress, or
surrender; and
(ii) following step (i), administering to the subject the psychedelic agent.

28. The method of claim 26 or 27, wherein the subject has been identified as likely to have a positive response to a psychedelic agent based on a low measure of:
mental barriers; or
preoccupation.

29. The method of any one of claims 1-28, wherein the psychedelic agent is administered as an adjunctive therapy, wherein the subject is being treated with a psychotherapy.

30. The method of any one of claims 1-19, further comprising instructing the subject to meditate.

31. The method of any one of claims 1-30, wherein step (ii) further comprises treating the subject with a psychotherapy.

32. The method of claim 31, wherein the psychotherapy comprises talk therapy.

33. The method of claim 31, wherein the psychotherapy comprises existential therapy.

34. The method of claim 31, wherein the psychotherapy comprises self-actualization therapy.

35. The method of any one of claims 1-34, wherein step (ii) occurs within a psychotherapeutic setting.

36. The method of claim 35, wherein the psychotherapeutic setting is a specialized treatment facility.

37. The method of any one of claims 1-36, wherein the positive therapeutic response to the psychedelic agent is preceded by a psychedelic-induced mystical experience (ME).

38. The method of any one of claims 5-37, wherein the psychological disorder is selected from the group consisting of a depressive disorder, an anxiety disorder, an addiction, or a compulsive behavior disorder.

39. The method of claim 38, wherein the psychological disorder is a depressive disorder.

40. The method of claim 39, wherein the depressive disorder is major depression, melancholic depression, atypical depression, or dysthymia.

41. The method of claim 39 or 40, wherein the depressive disorder is associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

42. The method of claim 38, wherein the psychological disorder is an anxiety disorder.

43. The method of claim 42, wherein said anxiety disorder is end of life anxiety, generalized anxiety disorder, panic disorder, social anxiety, post-traumatic stress disorder, acute stress disorder, obsessive compulsive disorder, or a social phobia.

44. The method of claim 38, wherein the psychological disorder is an addiction.

45. The method of claim 44, wherein the addiction is substance abuse or an eating disorder.

46. The method of claim 38, wherein the psychological disorder is a compulsive disorder.

47. The method of claim 46, wherein the compulsive behavior disorder is a primary impulse-control disorder or an obsessive-compulsive disorder.

48. The method of any one of claims 38-47, wherein a symptom of the psychological disorder is associated with a somatic symptom.

49. The method of claim 48, wherein the somatic symptom comprises chronic pain, anxiety disproportionate to severity of physical complaints, pain disorder, body dysmorphia, conversion, hysteria, neurological conditions without identifiable cause, or psychosomatic illness.

50. The method of any one of claims 5-49, wherein the psychological disorder is a repetitive body-focused behavior.

51. The method of claim 50, wherein the repetitive body-focused behavior is tic disorder.

52. The method of claim 51, wherein the tic disorder comprises Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder.

53. The method of any one of claims 1-52, wherein the psychedelic agent is selected from lysergic acid diethylamide, psilocybin, and pharmaceutically acceptable salts thereof.

54. A method of screening a subject for treatment with a psychedelic agent, the method comprising:
(i) providing a score of one or more predictors of a subject, wherein the score indicates whether a subject is likely to respond to a psychedelic agent; and
(ii) based on the score, identifying the subject as likely to respond to the psychedelic agent.

55. The method of claim 54, wherein the subject's response to the psychedelic agent is a positive therapeutic response.

56. The method of claim 54 or 55, wherein the one or more predictors comprises one or more trait predictors.

57. The method of claim 56, wherein the one or more trait predictors comprises a measure of absorption and/or a measure of mental barriers.

58. The method of claim 57, wherein a high measure of absorption is positively correlated with a positive response.

59. The method of claim 57 or 58, wherein a high measure of mental barriers is inversely correlated with a positive response.

60. The method of any one of claims 54-59, wherein the one or more predictors comprises one or more state predictors.

61. The method of claim 60, wherein the one or more state predictors are selected from the group consisting of a measure of distress, a measure of preoccupation, and a measure of surrender.

62. The method of claim 61, wherein a high measure of distress is positively correlated with a positive response.

63. The method of claim 61 or 62, wherein a high measure of preoccupation is inversely correlated with a positive response.

64. The method of any one of claims 61-63, wherein a high measure of surrender is positively correlated with a positive response.

65. The method of any one of claims 54-64, wherein the one or more predictors comprises one or more habits.

66. The method of claim 65, wherein the one or more habits comprises frequency of meditation.

67. The method of claim 66, wherein the frequency of meditation is positively correlated with a positive therapeutic response.

68. The method of claim 65, wherein the one or more habits comprises frequency of cannabis use.

69. The method of claim 68, wherein frequency of cannabis use is positively correlated with a positive therapeutic response.

70. The method of claim 54, wherein the one or more predictors are selected from the group consisting of a measure of absorption, a measure of mental barriers, a measure of meditation frequency, a measure of distress, a measure of preoccupation, and a measure of surrender.

71. The method of any one of claims 54-70, wherein the score is a composite score of at least two, at least three, at least four, at least five, or at least six of the predictors.

72. The method of claim 71, wherein the composite score is a weighted composite score.

73. The method of claim 72, wherein each predictor is weighted based on its explanatory power on positive therapeutic response to the psychedelic agent.

74. The method of claim 73, wherein the explanatory power is determined by a regression model.

75. The method of claim 74, wherein the regression model is a hierarchical linear multiple regression.

76. A method of screening a subject for treatment with a psychedelic agent, the method comprising:
(i) providing a score of one or more predictors of a subject, wherein the score reflects a high measure of absorption, a high measure of identity distress, and/or a high measure of surrender; and
(ii) based on the score, identifying the subject as likely to have a positive therapeutic response to the psychedelic agent.

77. The method of claim 76, wherein the score further reflects a low measure of mental barriers and/or a low measure of preoccupation.

78. The method of any one of claims 55-77, wherein the positive therapeutic response to the psychedelic agent is preceded by an ME.

79. The method of any one of claims 55-77, wherein the psychedelic agent is administered as an adjunctive therapy, wherein the subject is being treated with a psychotherapy.

80. The method of any one of claims 55-79, further comprising instructing the subject to meditate.

81. The method of any one of claims 55-80, wherein step (ii) further comprises treating the subject with a psychotherapy.

82. The method of claim 81, wherein the psychotherapy comprises talk therapy.

83. The method of claim 81, wherein the psychotherapy comprises existential therapy.

84. The method of claim 81, wherein the psychotherapy comprises self-actualization therapy.

85. The method of any one of claims 55-84, wherein step (ii) occurs within a psychotherapeutic setting.

86. The method of claim 85, wherein the psychotherapeutic setting is a specialized treatment facility.

87. The method of any one of claims 55-86, further comprising administering to the subject the psychedelic agent.

88. The method of claim 87, wherein the psychedelic agent is selected from the group consisting of a 5-HT_{2A} receptor agonist, an empathogenic agent, and a dissociative agent.

89. The method of claim 88, wherein the psychedelic agent is a 5-HT_{2A} receptor agonist.

90. The method of claim 89, wherein the 5-HT_{2A} receptor agonist is selected from lysergic acid diethylamide (LSD), psilocybin, DOI (±)-1-(2,5-dimethoxyphenyI)-2-aminopropane hydrochloride; (R)-DOI ((R)-1-(2,5-dimethoxy-4-iodophenyl)-2-aminopropane); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine) ayahuasca; 3,4,5-trimethoxyphenethylamine (mescaline); 5-methoxy-N,N-dimethyltryptamine (5-meo-DMT); ibogaine; a compound of 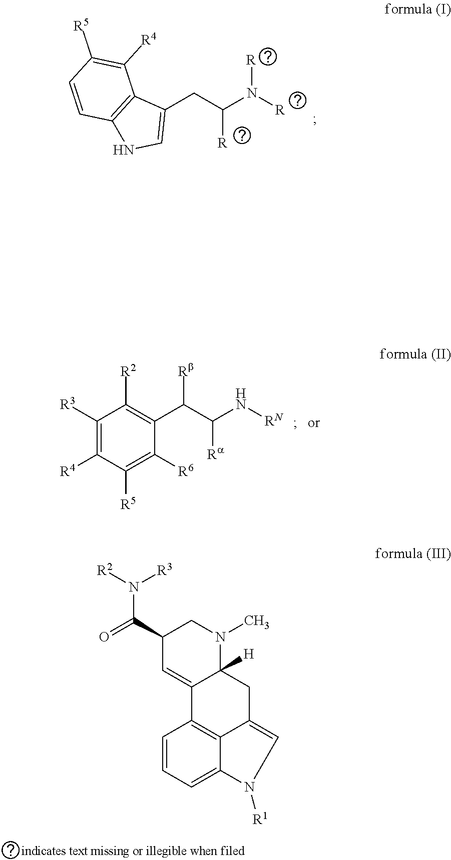
or a pharmaceutically acceptable salt thereof.

91. The method of claim 88, wherein the psychedelic agent is an empathogenic agent.

92. The method of claim 91, wherein the empathogenic agent is 3,4-Methylenedioxymethamphetmine (MDMA).

93. The method of claim 88, wherein the psychedelic agent is a dissociative agent.

94. The method of claim 93, wherein the dissociative agent is ketamine.

95. The method of any one of claims 54-94, wherein the score indicates that the subject is unlikely to have a positive response to the psychedelic agent.

96. The method of claim 95, wherein the one or more predictors comprises one or more trait predictors.

97. The method of claim 96, wherein the one or more trait predictors comprises a measure of mental barriers.

98. The method of claim 97, wherein a high measure of mental barriers is inversely correlated with a positive response.

99. The method of any one of claims 95-98, wherein the one or more predictors comprises one or more state predictors.

100. The method of claim 99, wherein the one or more state predictors are selected from the group consisting of a measure of distress, a measure of preoccupation, and a measure of surrender.

101. The method of claim 100, wherein a high measure of distress is inversely correlated with a positive response.

102. The method of claim 100 or 101, wherein a high measure of preoccupation is inversely correlated with a positive response.

103. The method of any one of claims 100-102, wherein a low measure of surrender is inversely correlated with a positive response.

104. The method of claim 95, wherein the one or more predictors are selected from the group consisting of a measure of absorption, a measure of mental barriers, a measure of meditation frequency, a measure of distress, a measure of preoccupation, and a measure of surrender.

105. The method of any one of claims 95-104, wherein the score is a composite score of at least two, at least three, at least four, at least five, or at least six of the predictors.

106. The method of claim 105, wherein the composite score is a weighted composite score.

107. The method of claim 106, wherein each predictor is weighted based on its explanatory power on positive response to the psychedelic agent.

108. The method of claim 107, wherein the explanatory power is determined by regression model.

109. The method of claim 108, wherein the regression model is a hierarchical linear multiple regression.

110. The method of any one of claims 95-109, wherein the subject is likely to have a psychedelic-induced negative experience.

111. A method of screening a subject for treatment with a psychedelic agent, the method comprising:
(i) providing a score of one or more predictors of a subject, wherein the score reflects a high measure of mental barriers, a high measure of identity distress, and/or a high measure of preoccupation; and
(ii) based on the score, identifying the subject as unlikely to have a positive response to the psychedelic agent.

112. The method of claim 111, wherein the score additionally reflects a low measure of absorption or a low measure of surrender.

113. The method of claim 111 or 112, further comprising the subject to meditate.

114. The method of any one of claims 111-113, wherein step (ii) further comprises treating the subject with a psychotherapy.

115. The method of claim 114, wherein the psychotherapy comprises talk therapy.

116. The method of claim 114, wherein the psychotherapy comprises existential therapy.

117. The method of claim 114, wherein the psychotherapy comprises self-actualization therapy.

118. The method of any one of claims 95-117, wherein step (ii) occurs within a psychotherapeutic setting.

119. The method of claim 118, wherein the psychotherapeutic setting is a specialized treatment facility.

120. The method of any one of claims 54-119, wherein the subject is being screened for treatment to improve the mental well-being of a subject.

121. The method of claim 120, wherein the subject is being screened for treatment of stress, treatment of anxiety, treatment of addiction, treatment of depression, or treating of a compulsive behavior.

122. The method of claim 120 or 121, wherein the subject is obese, and the subject is being screened for treatment for weight loss.

123. The method of claim 120, wherein the subject is being screened for treatment of a condition, improvement of mood, or enhancement of performance.

124. The method of claim 123, wherein the condition is a psychological disorder.

125. The method of claim 124, wherein the psychological disorder is selected from the group consisting of a depressive disorder, an anxiety disorder, an addiction, a compulsive behavior disorder, or a symptom thereof.

126. The method of claim 125, wherein the psychological disorder is a depressive disorder.

127. The method of claim 126, wherein the depressive disorder is major depression, melancholic depression, atypical depression, or dysthymia.

128. The method of claim 126 or 127, wherein the depressive disorder is associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

129. The method of claim 125, wherein the psychological disorder is an anxiety disorder.

130. The method of claim 129, wherein said anxiety disorder is end of life anxiety, generalized anxiety disorder, panic disorder, social anxiety, post-traumatic stress disorder, acute stress disorder, obsessive compulsive disorder, or a social phobia.

131. The method of claim 125, wherein the psychological disorder is an addiction.

132. The method of claim 131, wherein the addiction is substance abuse or an eating disorder.

133. The method of claim 125, wherein the psychological disorder is a compulsive disorder.

134. The method of claim 133, wherein the compulsive behavior disorder is a primary impulse-control disorder or an obsessive-compulsive disorder.

135. The method of any one of claims 125-134, wherein a symptom of the psychological disorder is associated with a somatic symptom.

136. The method of claim 135, wherein the somatic symptom comprises chronic pain, anxiety disproportionate to severity of physical complaints, pain disorder, body dysmorphia, conversion, hysteria, neurological conditions without identifiable cause, psychosomatic illness, pain management in relation to existing physical condition.

137. The method of any one of claims 124-136, wherein the psychological disorder is a repetitive body-focused behavior.

138. The method of claim 137, wherein the repetitive body-focused behavior is tic disorder.

139. The method of claim 138, wherein the tic disorder comprises Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder.

140. The method of claim 10, wherein the one or more state predictors are selected from the group consisting of a measure of motivations, a measure of distress, a measure of preoccupation, and a measure of surrender.

141. The method of claim 140, wherein a high measure of motivations is positively correlated with a positive therapeutic response.

142. The method of claim 140 or 141, wherein a high measure of surrender is positively correlated with a positive therapeutic response.

143. A method of improving the mental or physical well-being of a subject, the method comprising:
(i) providing a subject, wherein the subject has been identified as likely to have a positive therapeutic response to a psychedelic agent based on a high measure of:
absorption;
identity distress; or
motivations; and
(ii) following step (i), administering to the subject the psychedelic agent.

144. A method of reducing the risk of developing a psychological disorder in a subject in need thereof, the method comprising:
(i) providing a subject, wherein the subject has been identified as likely to have a positive therapeutic response to a psychedelic agent based on a high measure of:
absorption,
identity distress, or
motivations; and
(ii) following step (i), administering to the subject the psychedelic agent.

145. A method of screening a subject for treatment with a psychedelic agent, the method comprising:
(i) providing a score of one or more predictors of a subject, wherein the score reflects a high measure of absorption, a high measure of identity distress, and/or a high measure of motivations; and
(ii) based on the score, identifying the subject as likely to have a positive therapeutic response to the psychedelic agent.
